(19) United States
(12) Patent Application Publication
(10) Pub. No.: US 2026/0118308 A1
(43) Pub. Date: Apr. 30, 2026

(54) Rapid Point-of-Site Multiplexed Detection of Salivary Micro RNA in Mild TBI using Amplification-free Electrochemical Test

(71) Applicants: The Penn State Research Foundation, University Park, PA (US); UNIVERSITY OF MARYLAND, BALTIMORE COUNTY, Baltimore, MD (US); University of Maryland, Baltimore, Baltimore, MD (US)

(72) Inventors: Dipanjan Pan, University Park, PA (US); Parikshit Moitra, University Park, PA (US); Ketan Dighe, University Park, PA (US); Pranay Saha, University Park, PA (US); David Skrodzki, University Park, PA (US); Andre Van Der Vlies, University Park, PA (US)

(73) Assignees: UNIVERSITY OF MARYLAND, BALTIMORE COUNTY, Baltimore, MD (US); University of Maryland, Baltimore, Baltimore, MD (US); The Penn State Research Foundation, University Park, PA (US)

(21) Appl. No.: 19/165,875

(22) PCT Filed: Feb. 17, 2025
(86) PCT No.: PCT/US2025/016217

§ 371 Date: Sep. 16, 2025

Related U.S. Application Data
(60) Provisional application No. 63/555,944, filed on Feb. 21, 2024.

(51) Int. Cl. G01N 27/327; C12Q 1/6876

(52) U.S. Cl. CPC G01N 27/3276; C12Q 1/6876; C12Q 2600/112; C12Q 2600/178

(57) ABSTRACT

Embodiments relate to apparatuses, methods, and systems for screening and detecting mild traumatic brain injuries (mTBIs). Embodiments include an electrochemical sensor configured to receive and analyze a sample to determine if the sample includes at least one target gene sequence correlating to mTBI. The electrochemical sensor includes one more sensing probes functionalized on a polymer-based or COF-based conductive electrode.

### CROSS-REFERENCE TO RELATED APPLICATIONS

This patent application is related to and claims the benefit of priority of U.S. Provisional Application 63/555,944, filed on Feb. 21, 2024, the entire contents of which is incorporated by reference INCORPORATION BY REFERENCE STATEMENT REGARDING SEQUENCE LISTINGS

A Sequence Listing using exXtensible Markup Language (XML) compliant with World Intellectual Property Organization (WIPO) Standard ST.26 is provided herewith and the entirety of this sequence listing is incorporated by reference herein. The Sequence Listing that is incorporated by reference herein is provided via e-filing via ePCT as an XMVL file, named 0073605-000949.xml (4 KB in size, created on Feb. 14, 2025).

### FIELD

Embodiments relate to apparatuses, methods, and systems for screening and detecting a mild traumatic brain injury (mTBI). In particular, embodiments may relate to electrochemical sensors configured to detect the presence of one or more target gene sequences correlating to mTBI.

### BACKGROUND

Mild traumatic brain injuries (mTBIs) are prevalent and intricate neurological conditions, often manifesting in a range of symptoms that affect physical, cognitive, emotional, and sleep domains. The diagnostic challenge in mTBI stems from its subtle symptomatology and the lack of definitive biomarkers. Epidemiological data from the Centers for Disease Control and Prevention (CDC) indicate that between 1.7 and 3.8 million traumatic brain injuries (TBIs) occur annually in the United States, with a significant proportion affecting children and adolescents through sports and recreational activities. In these populations, over 21% of TBIs are linked to such activities. Moreover, injuries sustained in both military and civilian contexts are categorized by severity using the Glasgow Coma Scale (GCS), with immediate medical attention required for moderate-to-severe cases.

The lack of a standardized diagnostic test for mTBI leads to many cases going undiagnosed, exacerbating the long-term health and economic burden. mTBI is often associated with persistent symptoms like headaches, fatigue, concentration difficulties, and disruptions to daily activities. Current diagnostic standards primarily rely on clinical assessment, with neuroimaging techniques limited by cost, portability, and inability to detect subtle brain changes. The 2018 CDC guidelines emphasize the use of age-appropriate symptom rating scales and computerized cognitive tests but restrict the use of biomarkers to research contexts, highlighting the pressing need for more practical and reliable diagnostic tools.

Various technologies have been explored for TBI diagnosis, including computerized cognitive assessments, neuroimaging, electrophysiological methods, and serum-based biomarkers. However, these methods often face significant limitations. Computerized cognitive assessments are time-consuming, inconsistent, and expensive, hindering neuroimaging and electrophysiological methods, especially for younger individuals due to the need for specialized equipment. Serum-based biomarkers, such as S100 calcium-binding protein B, glial fibrillary acidic protein (GFAP), and ubiquitin C-terminal hydrolase (UCH-L1), have shown potential but are not yet validated for pediatric populations or practical for field use.

### SUMMARY

Recent research has identified microRNAs (miRNAs) as promising biomarkers for mTBI. These small, non-coding RNA molecules are involved in regulating gene expression and have been found to be dysregulated following neuronal injury. Notably, miRNAs such as miR-21, miR-30e, and let-7a exhibit altered expression levels in saliva samples from mTBI patients compared to healthy controls. Saliva, being non-invasive and readily accessible, presents a practical medium for miRNA-based diagnostics, avoiding the need for venipuncture and other invasive procedures.

Despite their potential, detecting miRNAs presents challenges due to their low abundance and instability in biological samples. Traditional methods for miRNA profiling, including northern blotting, reverse transcription polymerase chain reaction (RT-PCR), microarrays, and next-generation sequencing (NGS), are either too complex, costly, or time-consuming for routine clinical application. The need for high sensitivity and specificity in miRNA detection further complicates their use at the point of care. The dysregulation of salivary miRNAs following TBIs involves complex mechanisms, including altered transcriptional regulation, epigenetic changes, and post-transcriptional processes. miRNAs may also cross the blood-brain barrier and impact salivary gland function, complicating their detection and interpretation.

We have discovered a precise and innovative technique for detecting miRNAs in clinical samples, aiming to revolutionize mTBI diagnosis. For example, design of anti-miRNA oligonucleotides for three upregulated salivary miRNAs (miR-21, miR-30e, and let-7a) may be further modified and conjugated to an electrode to develop rapid, cost-effective, and non-invasive diagnostic methods. By this method, we seek to enhance early detection and intervention, improve patient outcomes, and reduce the overall healthcare burden of mTBI. Antisense oligonucleotide based detection can lead to highly precise disease diagnostics. Electrochemical detection through a conductive polymer-coated electrode, or a covalent organic framework (COF)-based conductive electrode, can be an excellent choice where the polymeric electrode surface can be modified according to a choice of detection.

Conductive polymers are specialized macromolecules that combine traditional polymers' structural and mechanical properties with the ability to conduct electricity. This unique feature makes them important for advanced electronics, energy storage, and sensing applications. Notable examples include polyaniline, polypyrrole, and poly(3,4-ethylenedioxythiophene) (PEDOT), which are integral to flexible electronics, organic light-emitting diodes (OLEDs), and photovoltaic cells due to their high electrical conductivity and environmental responsiveness. Conductive polymers, like PEDOT, are central in sensing technologies because they have the ability to convert stimuli into electrical signals, thus enhancing sensitivity and selectivity. They are often used to functionalize electrodes, optimizing sensor performance for disease diagnostics.

Further functionalization of conductive polymers, such as chemical modification with specific functional groups or conjugation with bio-recognition elements like antibodies or aptamers, may enhance specificity and enable selective interaction with target biomarkers. Such modifications may be crucial for stable, reproducible, and highly accurate disease diagnostics.

Moreover, nanoscale COFs offer high surface area, tunable porosity, and chemical stability, making them ideal for drug delivery, sensing, and catalysis. Post-synthetic modification (PSM) may introduce functional groups like target gene sequences and create multifunctional theragnostic platforms.

Accordingly, we have focused on engineering new, dynamically modifiable conductive polymer-based and COF-based electrochemical biosensors, paving the way for more precise disease diagnostics and improved healthcare outcomes. Embodiments may include a chemically modifiable, polymer-based or COF-based electrochemical biosensor for ultra-sensitive detection of miRNAs specific to mTBI.

In an exemplary embodiment, an apparatus for detecting a mild traumatic brain injury from a sample comprises an electrode comprising a polymeric layer coated on a surface of the electrode, wherein the polymeric layer includes at least one conductive polymer; and one or more sensing probes immobilized on the surface of the electrode, wherein the one or more sensing probes have a sequence that is complementary to a target gene sequence correlating to the mild traumatic brain injury, wherein a first end of the one or more sensing probes is functionalized with a molecule configured to facilitate immobilization to the surface of the electrode, and wherein a second end of the one or more sensing probes is functionalized with an electrochemical reporter configured to produce a measurable signal.

In an exemplary embodiment, the one or more sensing probes comprise a first sensing probe having a sequence that is complementary to a first target gene sequence correlating to the mild traumatic brain injury; and a second sensing probe having a sequence that is complementary to a second target gene sequence correlating to the mild traumatic brain injury.

In some embodiments, the first target gene sequence and the second target gene sequence are different sequences selected from the group consisting of UGAGGUAGUAGGUUGUAUAGU, UGUAAACAUCCUUGACUGGAAG, and UAGCUUAUCAGACUGAUGUUGA.

In some embodiments, the one or more sensing probes comprise a first sensing probe having a sequence that is complementary to a first target gene sequence correlating to the mild traumatic brain injury; a second sensing probe having a sequence that is complementary to a second target gene sequence correlating to the mild traumatic brain injury; and a third sensing probe having a sequence that is complementary to a third target gene sequence correlating to the mild traumatic brain injury.

In some embodiments, the first target gene sequence, the second target gene sequence, and the third target gene sequence are different sequences selected from the group consisting of UGAGGUAGUAGGUUGUAUAGU, UGUAAACAUCCUUGACUGGAAG, and UAGCUUAUCAGACUGAUGUUGA.

In some embodiments, the molecule is biotin.

In some embodiments, the electrochemical reporter is methylene blue.

In some embodiments, the at least one conductive polymer is selected from the group consisting of polystyrene sulfonate, poly(3,4-ethylenedioxythiophene), polypyrrole, polyamine, and combinations thereof.

In some embodiments, the electrode further includes a linking compound covalently attached to the polymeric layer and configured to facilitate immobilization of the one or more sensing probes to the surface of the electrode.

In some embodiments, the linking compound is streptavidin.

In some embodiments, the polymeric layer further includes at least one monomer polymerizable with the at least one conductive polymer.

In some embodiments, the at least one monomer is functionalized with carboxylic acid groups configured to facilitate attachment of the linking compound to the polymeric layer.

In some embodiments, the at least one monomer is a thiophene-based monomer functionalized with carboxylic acid groups configured to facilitate attachment of the linking compound to the polymeric layer.

In an exemplary embodiment, a method for detecting a mild traumatic brain injury comprises collecting a sample comprising nucleic acid from a subject; providing an apparatus comprising an electrode comprising a polymeric layer coated on a surface of the electrode, wherein the polymeric layer includes at least one conductive polymer, and one or more sensing probes immobilized on the surface of the electrode, wherein the one or more sensing probes have a sequence that is complementary to a target gene sequence correlating to the mild traumatic brain injury, wherein a first end of the one or more sensing probes is functionalized with a molecule configured to facilitate immobilization to the surface of the electrode, and wherein a second end of the one or more sensing probes is functionalized with an electrochemical reporter configured to produce a measurable signal; and applying the sample to the apparatus.

In some embodiments, the one or more sensing probes comprise a first sensing probe having a sequence that is complementary to a first target gene sequence correlating to the mild traumatic brain injury; and a second sensing probe having a sequence that is complementary to a second target gene sequence correlating to the mild traumatic brain injury.

In some embodiments, the first target gene sequence and the second target gene sequence are different sequences selected from the group consisting of UGAGGUAGUAGGUUGUAUAGU, UGUAAACAUCCUUGACUGGAAG, and UAGCUUAUCAGACUGAUGUUGA.

In some embodiments, the one or more sensing probes comprise a first sensing probe having a sequence that is complementary to a first target gene sequence correlating to the mild traumatic brain injury; a second sensing probe having a sequence that is complementary to a second target gene sequence correlating to the mild traumatic brain injury; and a third sensing probe having a sequence that is complementary to a third target gene sequence correlating to the mild traumatic brain injury.

In some embodiments, the first target gene sequence, the second target gene sequence, and the third target gene sequence are different sequences selected from the group consisting of UGAGGUAGUAGGUUGUAUAGU, UGUAAACAUCCUUGACUGGAAG, and UAGCUUAUCAGACUGAUGUUGA.

In an exemplary embodiment, an apparatus for detecting a mild traumatic brain injury from a sample comprises an electrode comprising a first polymeric layer, a covalent organic framework (COF) layer, and a second polymeric layer deposited on a surface of the electrode; and one or more sensing probes immobilized on the surface of the electrode, wherein the one or more sensing probes have a sequence that is complementary to a target gene sequence correlating to the mild traumatic brain injury, wherein a first end of the one or more sensing probes is functionalized with a molecule configured to facilitate immobilization to the surface of the electrode, and wherein a second end of the one or more sensing probes is functionalized with an electrochemical reporter configured to produce a measurable signal.

In some embodiments, the one or more sensing probes comprise a first sensing probe having a sequence that is complementary to a first target gene sequence correlating to the mild traumatic brain injury; and a second sensing probe having a sequence that is complementary to a second target gene sequence correlating to the mild traumatic brain injury.

In some embodiments, the first target gene sequence and the second target gene sequence are different sequences selected from the group consisting of UGAGGUAGUAGGUUGUAUAGU, UGUAAACAUCCUUGACUGGAAG, and UAGCUUAUCAGACUGAUGUUGA.

In some embodiments, the one or more sensing probes comprise a first sensing probe having a sequence that is complementary to a first target gene sequence correlating to the mild traumatic brain injury; a second sensing probe having a sequence that is complementary to a second target gene sequence correlating to the mild traumatic brain injury; and a third sensing probe having a sequence that is complementary to a third target gene sequence correlating to the mild traumatic brain injury.

In some embodiments, the first target gene sequence, the second target gene sequence, and the third target gene sequence are different sequences selected from the group consisting of UGAGGUAGUAGGUUGUAUAGU, UGUAAACAUCCUUGACUGGAAG, and UAGCUUAUCAGACUGAUGUUGA.

In some embodiments, the molecule is biotin.

In some embodiments, the electrochemical reporter is methylene blue.

In some embodiments, the polymeric layer includes at least one conductive polymer.

In some embodiments, the at least one conductive polymer is selected from the group consisting of polystyrene sulfonate, poly(3,4-ethylenedioxythiophene), polypyrrole, polyamine, and combinations thereof.

In some embodiments, the COF layer is positioned between the first polymeric layer and the second polymeric layer.

In some embodiments, the electrode further includes a linking compound covalently attached to the COF layer and configured to facilitate immobilization of the one or more sensing probes to the surface of the electrode.

In some embodiments, the linking compound is streptavidin.

In some embodiments, the COF layer is functionalized with epoxy groups configured to facilitate attachment of the linking compound to the COF layer.

In an exemplary embodiment, a method for detecting a mild traumatic brain injury the method comprises collecting a sample comprising nucleic acid from a subject; providing an apparatus comprising an electrode comprising a first polymeric layer, a covalent organic framework (COF) layer, and a second polymeric layer deposited on a surface of the electrode, and one or more sensing probes immobilized on the surface of the electrode, wherein the one or more sensing probes have a sequence that is complementary to a target gene sequence correlating to the mild traumatic brain injury, wherein a first end of the one or more sensing probes is functionalized with a molecule configured to facilitate immobilization to the surface of the electrode, and wherein a second end of the one or more sensing probes is functionalized with an electrochemical reporter configured to produce a measurable signal; and applying the sample to the apparatus.

In some embodiments, the one or more sensing probes comprise a first sensing probe having a sequence that is complementary to a first target gene sequence correlating to the mild traumatic brain injury; and a second sensing probe having a sequence that is complementary to a second target gene sequence correlating to the mild traumatic brain injury.

In some embodiments, the first target gene sequence and the second target gene sequence are different sequences selected from the group consisting of UGAGGUAGUAGGUUGUAUAGU, UGUAAACAUCCUUGACUGGAAG, and UAGCUUAUCAGACUGAUGUUGA.

In some embodiments, the one or more sensing probes comprise a first sensing probe having a sequence that is complementary to a first target gene sequence correlating to the mild traumatic brain injury; a second sensing probe having a sequence that is complementary to a second target gene sequence correlating to the mild traumatic brain injury; and a third sensing probe having a sequence that is complementary to a third target gene sequence correlating to the mild traumatic brain injury.

In some embodiments, the first target gene sequence, the second target gene sequence, and the third target gene sequence are different sequences selected from the group consisting of UGAGGUAGUAGGUUGUAUAGU, UGUAAACAUCCUUGACUGGAAG, and UAGCUUAUCAGACUGAUGUUGA.

Other details, objects, and advantages of our apparatuses for screening and detecting mTBIs, methods for screening and detecting mTBIs, and systems for screening and detecting mTBIs will become apparent as the following description of certain exemplary embodiments thereof proceeds.

### BRIEF DESCRIPTION OF THE DRAWINGS

The above and other objects, aspects, features, advantages, and possible applications of embodiments of the present innovation will be more apparent from the following more particular description thereof, presented in conjunction with the following drawings. Like reference numbers used in the drawings may identify like components.

FIG. 1 shows a schematic representation of an exemplary method for screening and detecting mTBI. The schematic representation demonstrates a change in electrical output upon hybridization of one or more target sequences with sensing probes functionalized on polymer coated electrodes.

FIG. 2 shows a schematic representation of an exemplary system for screening and detecting mTBI. The schematic representation shows an exemplary detection procedure with an electrochemical sensor.

FIG. 3 shows a schematic representation of an exemplary polymer coated electrode. The schematic representation demonstrates streptavidin's physical adsorption and covalent immobilization to an electrode.

FIG. 4 shows a schematic representation of an exemplary post-synthetic modification of a covalent organic framework (COF) for enhanced functionalization of polymer coated electrodes.

FIG. 5 shows a schematic representation of an exemplary layered assembly of polymer coated electrodes. The schematic representation demonstrates an enhanced electrochemical current output in cyclic voltammetry (CV).

FIG. 6 shows a schematic representation of an exemplary mechanism of change in electrical output.

FIG. 7 is a block diagram illustrating an exemplary system for screening and detecting mTBI.

FIG. 8A is a table of target gene sequences (e.g., miRNA) correlating to mTBI.

FIG. 8B is a table of exemplary sensing probes with sequences complementary to the miRNA included in FIG. 8A.

FIG. 9 is a schematic representation of an exemplary synthesis scheme for an AAOT monomer and AAOT:PSS polymer. A chronological diagram is included at the bottom of the schematic representation.

FIG. 10 shows an infrared (IR) spectrum of AAOT in the spectral range from 3500-700 and 2100-700 cm⁻¹ showing the characteristic ν (C=O) stretching vibration of the carboxylate at around 2500-3300, 1700 cm⁻¹.

FIG. 11 shows a UV-Vis spectra of AAOT:PSS before (top) and after adding hydrazine (bottom). The inset shows a color change observed after treating AAOT:PSS with hydrazine.

FIG. 12 is a mechanistic representation of reduction in PEDOT and AAOT upon an electrochemical or hydrazine-based reduction.

FIG. 13 includes high magnification scanning electron microscopy (SEM) images showing an Au electrode and an AAOT:PSS Au electrode.

FIG. 14 includes low magnification SEM images showing an AAOT:PSS Au electrode and the electrode after covalent functionalization with streptavidin via EDC/NHS coupling chemistry.

FIGS. 15-17 are graphs showing CV measurements of miRNAs on anti-miRNA functionalized AAOT:PSS-Au electrodes exhibiting linear change in electrical output upon increasing concentrations of corresponding miRNAs.

FIG. 18 is a graph showing a difference in fluorescence (FL) intensity of covalently conjugated Streptavidin-Cy3 on AAOT:PSS and loosely attached on PEDOT: PSS after a single wash.

FIG. 19 is a graph showing an increased change in current output of electrodes upon functionalization with AAOT:PSS.

FIG. 20 is a graph showing consistent current output of AAOT:PSS-strp-ASO-MB up to 60 minutes after hybridization between anti-miRNA and miRNAs.

FIG. 21 is a graph showing long-term stability of the PEDOT:PSS-ASO-MB and AAOT:PSS-ASO-MB up to 30 days.

FIG. 22 is a graph demonstrating that saliva may show minimal matrix effect in CV, which does not affect measurements significantly.

FIG. 23 is a graph showing differences between miRNA levels for positive and negative patient samples.

FIG. 24 is a confusion matrix comparing electrochemical and polymerase chain reaction (PCR) test results.

FIG. 25 is a schematic representation showing a change in fluorescence of methylene blue-miR-ASO upon hybridization with corresponding miRNAs.

FIG. 26 shows graphs demonstrating that change in absorbance of MB-miR-ASO in saliva upon addition of an increasing amount of respective miRNA exhibits linear decrease.

FIG. 27 shows graphs demonstrating that change in fluorescence of MB-miR-ASO in saliva with increasing concentrations of respective miRNA exhibits a decrease in fluorescence intensity.

FIG. 28 shows an exemplary synthesis scheme for phloroglucinol and keto forms of the covalent organic framework in an organic nonpolar solvent.

FIG. 29 is a plot showing the change in ¹H NMR peak on aldehyde to hydroxyl transformation upon keto to enol tautomerization of COF from due to change in solvent from chloroform to dimethyl sulfoxide (DMSO).

FIG. 30 is a graph showing Fourier transform infrared (FTIR) spectroscopy peaks of specific bonds exhibiting formation of covalent organic framework from the monomers.

FIG. 31 is a graph showing thermogravimetric analysis demonstrates weight loss of the COF from 22° C. to 800° C. with high stability till 350° C.

FIG. 32 shows transmission electron microscopic images demonstrating monolayer and multilayer accumulation of silane-modified COF nanosheets with faintly visible lattice fringes.

FIG. 33 is a graph showing change and shift in Raman peak positions and intensities for Cy-streptavidin conjugation on functionalized electrode to determine the concentration of streptavidin attachment over the COF-Nafion-PEDOT-based electrode as depicted from solid state fluorescence spectroscopy.

FIG. 34 is a graph showing a standard curve for Cy-streptavidin conjugation on a functionalized electrode to determine the concentration of streptavidin attachment over the COF-Nafion-PEDOT-based electrode as depicted from solid state fluorescence spectroscopy.

FIG. 35 is a graph showing a differential conjugation profile of Cy3-streptavidin over different layers of functionalized electrodes.

FIG. 36 shows confocal microscopy images exhibiting differential distribution patterns of Cy-3 streptavidin over modified electrode surfaces (scale: 50 μM).

FIG. 37 shows graphs demonstrating that miRNA let7a exhibits linear change in current with increasing concentrations of miRNAs.

FIG. 38 shows graphs demonstrating that miR21 exhibits linear change in current with increasing concentrations of miRNAs.

FIG. 39 shows graphs demonstrating that miR30 exhibits linear change in current with increasing concentrations of miRNAs.

FIG. 40 is a graph showing long-term stability of the COF-Nafion-ASO-MB electrodes for up to 60 days.

FIG. 41 is a graph showing increased current output of electrodes upon functionalization with polymer layers.

FIG. 42 is a graph showing increased ionic conductivity with addition of polymer layers upon hybridization of anti-miRNA and miRNA.

FIG. 43 is a graph showing consistent current output on COF-Nafion-PEDOT-ASO-MB up to 60 minutes after hybridization between the anti-miRNA and miRNAs.

FIG. 44 is a graph demonstrating that saliva shows minimal matrix effect in CV which does not affect the measurements significantly.

FIG. 45 is a graph showing concentrations of miRs in patients' saliva samples incorporating calculation for concentration factor during miRNA extraction, depicted as per standard curve obtained from CV spectra for corresponding miR concentrations.

FIG. 46 is a graph showing the difference between miRNA levels for positive and negative patient samples.

FIG. 47 is a confusion matrix for comparing electrochemical and PCR results.

FIG. 48 is a Bland-Altman plot for correlating the data obtained from electrochemical and PCR results.

### DETAILED DESCRIPTION

The following description is of exemplary embodiments and methods of use that are presently contemplated for carrying out the present invention. This description is not to be taken in a limiting sense, but is made merely for the purpose of describing the general principles and features of various aspects of the present invention. The scope of the present invention is not limited by this description.

Embodiments generally relate to apparatuses, methods, and systems configured to accurately screen and detect mild traumatic brain injuries (mTBIs). Exemplary apparatuses, methods, and systems comprise sensing probes configured to selectively detect a target gene sequence (e.g., biomarker) correlating to mTBIs.

As used herein, the term “mild traumatic brain injury” or “mTBI” may refer to any traumatically induced physiological disruption of brain function. The terms “mild traumatic brain injury” and “traumatic brain injury” may be used interchangeably herein.

Embodiments relate to electrochemical sensors configured to receive and analyze a sample to determine if the sample includes at least one target gene sequence correlating to mTBI. The sensor includes one more sensing probes functionalized on an electrode. More specifically, one or more sensing probes may be immobilized on a surface of the electrode.

In some embodiments, the sensor can be used as a point-of-care (POC) test or point-of-site (POS), for example, as a rapid lab test, for screening and detecting mTBI.

In exemplary embodiments, the electrode is configured to detect an electrochemical signal (e.g., a redox signal, such as a current shift) in response to specific binding of a sensing probe and a corresponding target gene sequence present in a sample of a subject. In some embodiments, the electrode is in fluid contact with the sample after the sample is deposited onto the electrode.

The electrode may include any suitable electrode material. In some embodiments, the electrode may include a plasmonic material, such as gold, silver, and/or combinations thereof. For example, the electrode may be a gold electrode, a silver electrode, an electrode that includes gold, an electrode that includes silver, etc. In some embodiments, the electrode can be a carbon or graphene electrode. Other embodiments can utilize other types of electrode materials.

In some embodiments, the electrode can be a screen printed electrode wherein an ink is printed on a substrate. The ink may include a plasmonic material and/or other electrode materials.

In some embodiments, mediators (e.g., small molecules, nanomaterials, polymers, covalent organic frameworks, etc.) can be introduced to the electrode. The mediators may be configured to assist in the transfer of electrons to the electrode.

The electrode may include a first polymeric layer including at least one conductive polymer. The first polymeric layer may be coated on a surface of the electrode. The conductive polymer(s) may be selected from the group consisting of polystyrene sulfonate (PSS), poly(3,4-ethylenedioxythiophene) (PEDOT), polypyrrole (PPy), polyaniline (PANI), and mixtures thereof. Other embodiments may utilize another conductive polymer as well. The first polymeric layer may include any number of different conductive polymer(s), such as one or more conductive polymers, or a plurality of conductive polymers. In one embodiment, the at least one conductive polymer includes PEDOT:PSS.

The first polymeric layer may further include at least one monomer. The at least one monomer may be integrated with the conductive polymer(s), such that the at least one monomer is preferably polymerizable with the conductive polymer(s). In some embodiments, the at least one monomer may be functionalized with carboxylic acid groups. The carboxylic acid groups may be free to enable post-synthetic attachment of further compounds/molecules to the electrode.

In some embodiments, the monomer may be a thiophene-based monomer, such as AAOT (e.g., acid oxy thiophene).

In alternative embodiments, the electrode may include one or more, including the first polymeric layer and/or a covalent organic framework (COF) layer and/or a second polymeric layer. The COF layer may be deposited on a surface of the electrode, or may deposited on another layer. It is contemplated that the COF is a three-dimensional structure with a large surface area. In some embodiments, the COF may be based on p-phenylenediamine and 1,3,5-triformylphloroglucinol. The COF may be modified to include epoxy groups. The epoxy groups may be free to enable post-synthetic attachment of further compounds/molecules to the electrode. In some embodiments, the COF may be modified with (3-glycidoxypropyl)trimethoxy silane (GPTMS) to add epoxy groups.

The second polymeric layer may be coated on a surface of the electrode, or may be deposited on another layer. In some embodiments, the second polymeric layer may form a polymeric bilayer together with the first polymeric layer. In particular, a polymeric bilayer including the COF positioned between the second polymeric layer and the first polymeric layer may be formed. In some embodiments, the second polymeric layer may exhibit a high proton conductivity, ion selectively, and/or chemical stability. In some embodiments, the second polymeric layer may include Nafion or other materials demonstrating similar properties.

In some embodiments, a linking compound may be covalently attached to the electrode, more specifically to one or more layers deposited on the electrode. The linking compound is configured to facilitate binding and immobilization of the one or more sensing probes to the surface of the electrode. The linking compound may therefore be any compound or molecule able to facilitate such binding and immobilization. In some embodiments, the linking compound may be attached to the polymeric layer via a carboxylic group of at least one monomer, or to the COF via an epoxy group.

In some embodiments, the linking compound may be an amine-containing biomolecule, such as streptavidin.

In exemplary embodiments, the electrochemical sensor is configured to screen and detect mTBI. In particular, the electrochemical sensor utilizes one or more sensing probes to detect the presence of corresponding target gene sequences correlating to mTBI. Each sensing probe is designed specifically to bind in complementary fashion to a target gene sequence correlating to mTBI.

The one or more sensing probes may be anti-sense oligonucleotides (ASOs) configured to detect corresponding target gene sequences. The ASOs are designed specifically to bind in complementary fashion to a target gene sequence related to mTBI. For example, ASOs have nucleotide sequences that complement the nucleotide sequence of a target gene (e.g., adenine (A) in an ASO sequence may complement and bind to uracil (U) or thymine (T) in a target gene sequence, cytosine (C) in an ASO sequence may complement and bind to guanine (G) in a target gene sequence, thymine (T) or uracil (U) in an ASO sequence may complement and bind to adenine (A) in a target gene sequence, and guanine (G) in an ASO sequence may complement and bind to cytosine (C) in a target gene sequence). It is contemplated that the terms “anti-sense oligonucleotides” and “sensing probes” may be used interchangeably herein.

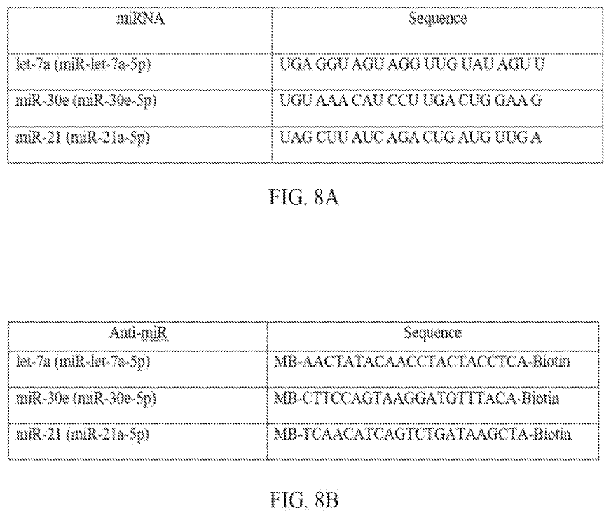

Accordingly, to screen and detect mTBI, at least one target gene sequence correlating to mTBI and brain injury mechanisms must first be identified such that sensing probes can be designed to complement and bind to the sequence. In some embodiments, a target gene sequence relating to mTBI may be chosen from SEQ ID NO 1 (UGA GGU AGU AGG UUG UAU AGU U), SEQ ID NO 2 (UGU AAA CAU CCU UGA CUG GAA G), and SEQ ID NO 3 (UAG CUU AUC AGA CUG AUG UUG A) (see FIGS. 8A and 8B).

SEQ ID NO 1 specifically relates to let7A miRNA, which plays a role in cell differentiation and proliferation. SEQ ID NO 2 specifically relates to miR-30e miRNA, which is linked to neuronal apoptosis and cellular stress response. SEQ ID NO 3 specifically relates to miR-20 miRNA, which is involved in regulating inflammation and apoptosis.

Sensing probes may then be designed to bind in complementary fashion to target gene sequences related to mTBI. For example, a sensing probe may have a sequence chosen from a sequence complementary to and configured to bind to SEQ ID NO 1, a sequence complementary to and configured to bind to SEQ ID NO 2, and complementary to and configured to bind to SEQ ID NO 3.

The sensing probes may be functionalized/modified at one end (e.g., a first end) with a first molecule. The first molecule may be configured to bind the sensing probes to the electrode. For example, the first molecule may bind to the linking compound, such that the sensing probe is immobilized on a surface of the electrode. In some embodiments, the first molecule may be biotin, cyclodextrins, cucurbituril, or caloxarene. In one embodiment, the first molecule is biotin.

In some embodiments, the sensing probes may be functionalized/modified at an opposite end (e.g., a second end) with a second molecule. The second molecule is configured to serve as an electrochemical reporter, such that it may provide a measurable redox signal (e.g., current shift) for monitoring target gene sequence hybridization. In particular, the second molecule is configured to produce measurable signals in response to conformational changes or variations in intramolecular distances when hybridization occurs between a sensing probe and a corresponding target gene sequence. In some embodiments, the second molecule may be methylene blue.

The sensing probes may be functionalized on the electrode in any configuration. In some embodiments, the sensing probes may form a hairpin loop structure.

A sensor may include any number of sensing probes. A sensor may include at least a first sensing probe configured to complement and bind to a first target gene sequence, a second sensing probe configured to complement and bind to a second target gene sequence, a third sensing probe configured to complement and bind to a third target gene sequence, etc.

In some embodiments, the sensor is configured to detect the presence of a predetermined target gene sequence. For example, the system may be configured to detect one of let7A, miR-30e, or mir-20.

In some embodiments, the sensor is configured to detect the presence of one or both of two predetermined target gene sequences. For example, the system may be configured to detect one or both of let7A and miR-30e, one or both of mir-20 and miR-30e, or one or both of let7A and miR-20.

In some embodiments, the sensor is configured to detect the presence of one, two, or all of three predetermined target gene sequences. For example, the system may be configured to detect one, two, or all of let7a, miR-30e, and mir-20.

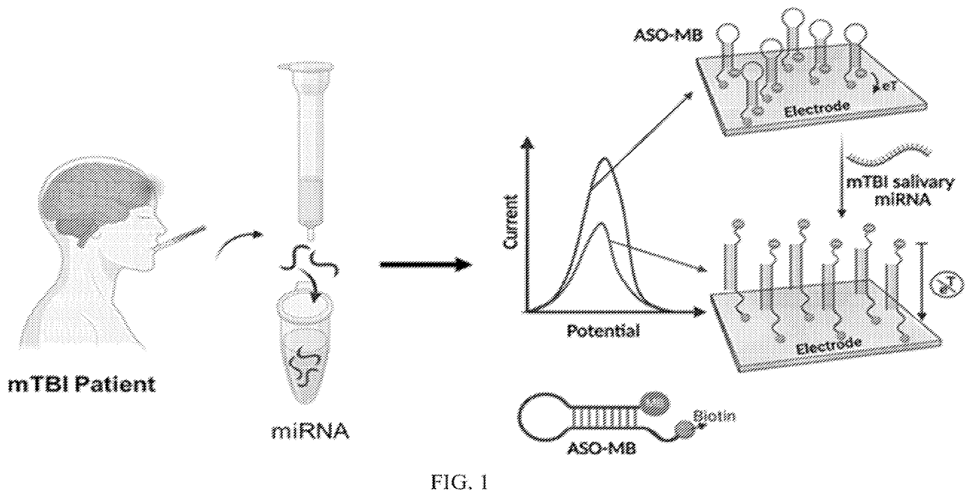

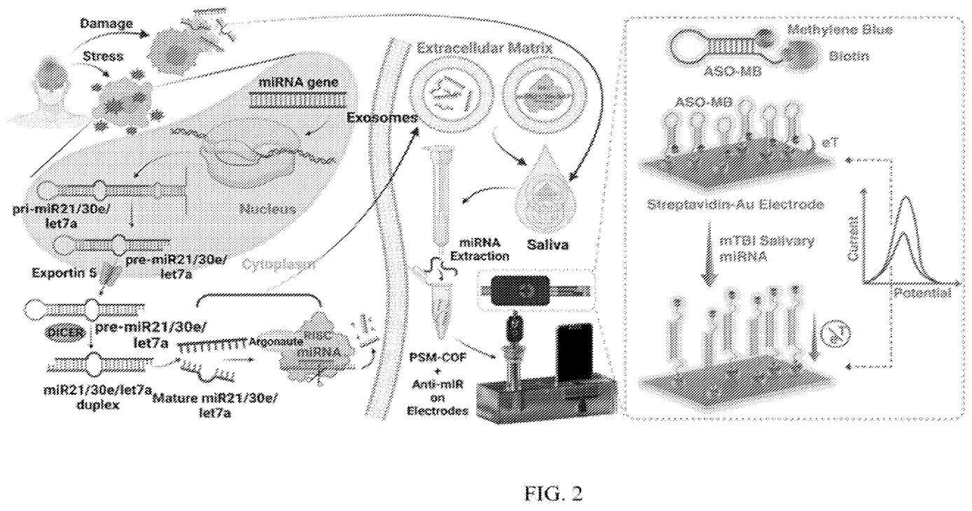

Referring to FIGS. 1 and 2, exemplary methods and systems for screening and detecting mTBI may comprise collecting a sample (e.g., an RNA sample) from a subject. It is contemplated that the sample may be collected using any suitable means, including but not limited to, an oral swab, a nasal swab, a cervical swab, a blood collecting swab, urine collection, or any other suitable means for collecting nucleic acid from the subject. In a preferred embodiment, the sample may be saliva, as it is non-invasive and can be collected easily.

It is further contemplated that the sample may be collected using any suitable instrument, including but not limited to, a cotton swab or any other suitable instrument for collecting nucleic acid from the subject.

The collected sample may then be introduced to a sensing solution to form an aqueous mixture. In some embodiments, the sensing solution may include a nucleic acid extraction buffer configured to extract nucleic acids from the collected sample. In alternative embodiments, nucleic acids may not be extracted from the collected sample prior to application on the electrode. Extraction of nucleic acid and amplification of nucleic acid may be performed but are not requirements for using the electrochemical sensor, thus allowing sensing of a target gene sequence directly from the collected sample.

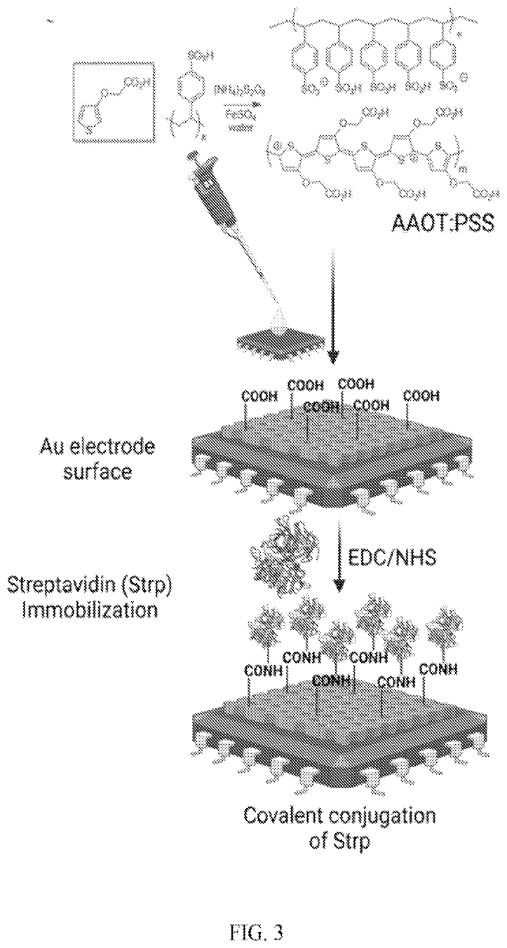

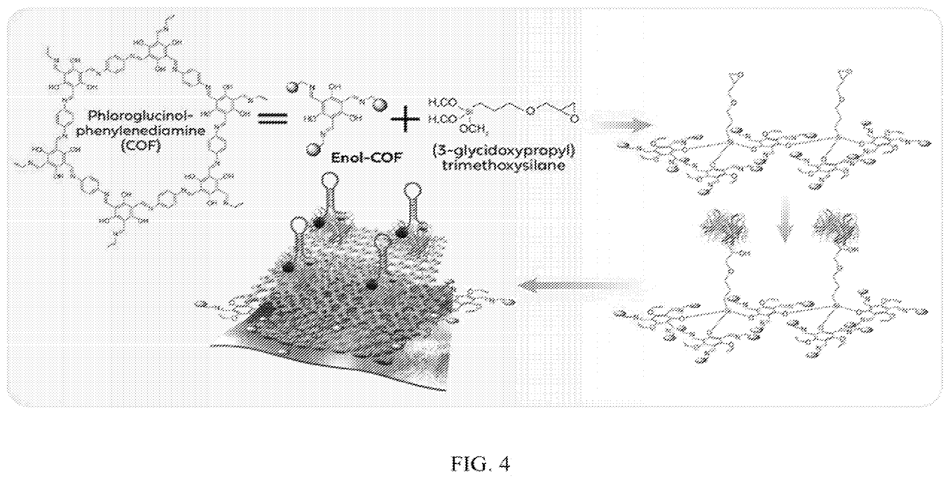

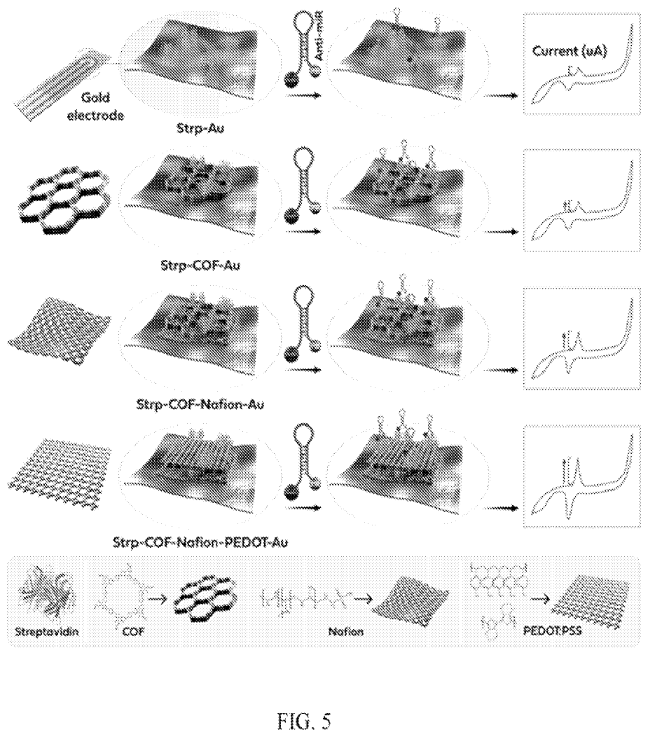

As can be appreciated from FIGS. 1-6, the aqueous mixture may then be deposited onto the surface of the electrochemical sensor (e.g., onto the electrode). The sensor includes one more sensing probes functionalized on the electrode. The electrode is configured to detect an electrochemical signal in response to specific binding of a sensing probe and a corresponding target gene sequence in the sample of a subject. In some embodiments, the electrochemical signal is a cyclic voltammetry (CV) signal, a differential pulse voltammetry (DPV) signal, or any other suitable signal. In some embodiments, the electrochemical signal, for example a redox current flowing through the electrode and/or an electrical impedance across the electrode, is in response to specific binding of a sensing probe and a corresponding target gene sequence in a sample of a subject.

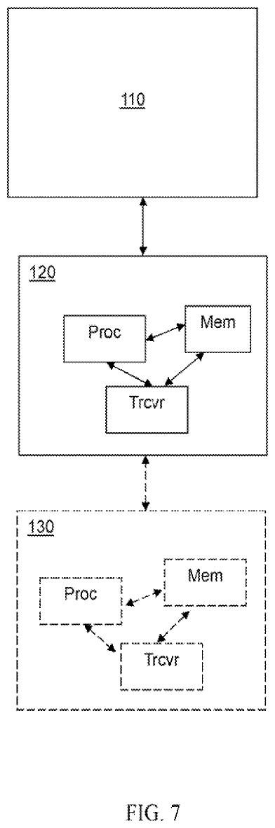

Referring to FIG. 7, the sensor 100 can be hardwire connected to an input/output device 110 (e.g., a smart phone, tablet, laptop computer, personal computer, computer device of a drone or robotic device, etc.), or the sensor 100 can be communicatively connected to the input/output device 110 via a network connection or wireless connection (e.g., internet connection, wide area network connection, near field communication connection, Bluetooth connection, etc.).

In some embodiments, the input/output device 110 can be configured to receive data from the sensor 100 for storage and analysis. In some implementations, the input/output device 110 can be configured as a server or cloud-based service providing device for storage and analysis of the data obtained via the sensor 100. The data can be communicated to a user via display device, which can be a tablet, smart phone, laptop computer, personal computer, or other type of terminal device. The display device can be effectuated via an application programming interface (API) and/or use of an application stored on the display device. It is contemplated that the input/output device 110 can comprise the display device, or the display device can be a separate device.

In some embodiments, the sensor 100 can alternatively or subsequently be sent to a central computer device 120 (e.g., a server, an operator workstation, etc.) that can be hardwire connected to the sensor 100 and/or the input/output device 110, or can be communicatively connected to the sensor 100 and/or the input/output 110 device via a network connection and/or a wireless connection. The central computer device 120 can be configured to store, analyze, and/or display data received from the sensor 100 and/or input/output device 110.

In some embodiments, the collected data can be continuously streamed to the input/output device 110 and/or the central computer device 120. In other embodiments, the collected data can be periodically streamed to the input/output device 110 and/or the central computer device 120 (e.g., non-continuously at pre-determined intervals). Embodiments of the sensor 100 can be configured to provide real time data collection.

The input/output device 110 and/or the central computer device 120 can be a computer device that can include a processor (Proc.) connected to a non-transitory memory (Mem.) and at least one transceiver (Trcvr) for forming communicative connections with one or more other devices. The at least one transceiver (Trcvr) can include a Bluetooth module and/or other type of transceiver unit (Trcvr). The processor can be hardware (e.g., processor, integrated circuit, central processing unit, microprocessor, core processor, computer device, etc.), configured to perform operations by execution of instructions embodied in algorithms, data processing program logic, artificial intelligence programming, automated reasoning programming, etc. that can be defined by code stored in the memory. The processor can facilitate receipt, processing, and/or storage of readings from the sensor 100 and/or control transmission of the collected data to input/output device 110 and/or the central computer device 120.

It should be noted that use of processors herein can include hardware, such as for example any one or combination of a Graphics Processing Unit (GPU), a Field Programmable Gate Array (FPGA), a Central Processing Unit (CPU), a microprocessor, a processor, etc. The processor can include one or more processing or operating modules. A processing or operating module can be a software or firmware operating module configured to implement any of the functions disclosed herein. The processing or operating module can be embodied as software and stored in non-transitory memory, the memory being operatively associated with the processor. A processing module can be embodied running a web application, a desktop application, a console application, etc.

The memory (Mem.) can be a non-transitory computer readable memory configured to store data. Embodiments of the memory can include a processor module and other circuitry to allow for the transfer of data to and from the memory, which can include to and from other components of a communication system. This transfer can be via hardwired links or wireless transmission communication links. The communication system can include transceivers, which can be used in combination with switches, receivers, transmitters, routers, gateways, waveguides, etc. to facilitate communications between different devices via a communication approach or protocol for controlled and coordinated signal transmission and processing to any other component or combination of components of the communication system. The transmission can be via a communication link, which can be a wireless type of communication connection and/or a wired type of connection.

The computer or non-transitory machine-readable medium can be configured to store one or more instructions thereon. The instructions can be in the form of algorithms, program logic, etc. that cause the processor to execute any of the functions disclosed herein.

The processor can be in communication with other processors of other devices (e.g., additional external device, a computer system, a laptop computer, a desktop computer, etc.). An exemplary other device can be a Bluetooth enabled device, near field communication device, etc. Any of those other devices can include any of the exemplary processors disclosed herein as well as transceivers or other communication devices/circuitry to facilitate transmission and reception of wireless signals or other type of communicative connections.

Either the input/output device 110 and/or the central computer device 120 can be configured to be connected to other input devices and output devices. Examples of input devices can include a scanner device (e.g., scanner), a microphone, a keyboard, a touch screen, a button, a sensor a detector, or other type of input device. Examples of output devices can include a display, a printer, a speaker, or other type of output device.

As noted above, once collected data is transmitted to the input/output device 110 and/or the central computer device 120, the data can be analyzed and evaluated to determine whether a subject has experienced mTBI. For example, electrochemical signals may be processed to determine if one or more target gene sequences are present in a sample collected from a subject. The signals may be processed to determine the presence or absence of target gene sequences in a samples.

### EXAMPLES

### Example 1: Polymer-Based Conductive Electrode and Electrochemical Sensor

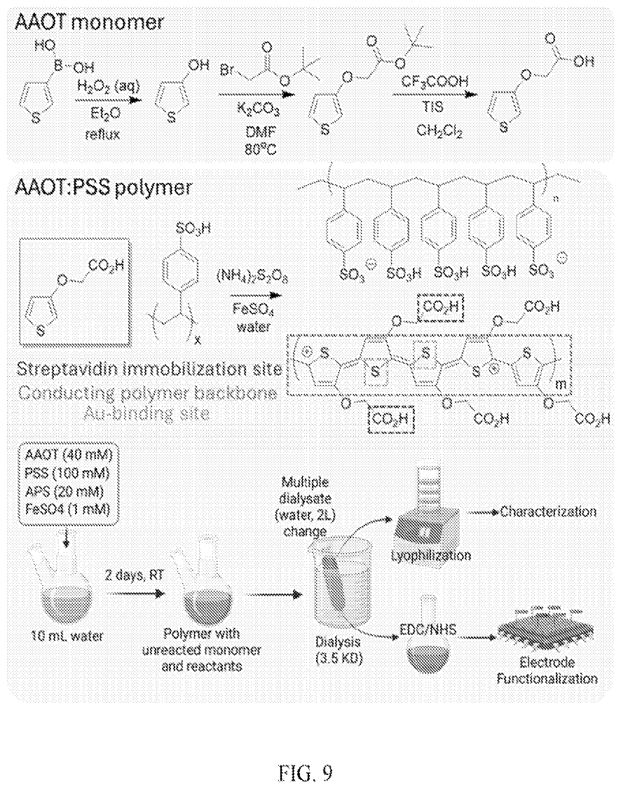

Methods and Materials—Synthesis of AAOT:PSS: The AAOT monomer was polymerized in the presence of polystyrene sulfonic acid (PSS) at room temperature using FeSO₄/(NH₄)₂S₂O₈(APS) as the initiation system. AAOT, PSS and APS were dissolved in water and polymerization was started by adding an aqueous solution of FeSO₄. Final concentrations: AAOT: 40 mM, PSS: 100 mM, APS: 20 mM, FeSO₄: 0.1. 1.0 or 10 mM corresponding to Fe/AAOT molar ratios of 0.0025, 0.025, 0.25. Polymerization was done for 18 h. UV-Vis spectra of the reaction mixtures (150 μL) were measured before and after adding 10 μL N₂H₄-H₂O in a 96 polystyrene well plate (FIG. 9).

Methods and Materials—Synthesis of AAOT:PSS for electrode functionalization: A volume of 10 mL water containing AAOT (40 mM), PSS (100 mM), APS (20 mM) and FeSO₄ (1.0 mM, 0.025 eq relative to AAOT) was stirred for 2 days at room temperature. The mixture was transferred to a dialysis tube (molecular weight cut of 3500) and dialyzed against 2 L water with regularly replacing the water. Part of the solution was lyophilized to determine the concentration of the polymer solution.

Methods and Materials—Preparation of NHS-activated AAOT:PSS: The free carboxyl groups of AAOT:PSS were activated using carbodiimide coupling chemistry using 1-ethyl-3-(3-dimethylaminopropyl)carbodiimide (EDC) and N-hydroxysuccinimide (NHS). To 1 mL of AAOT:PSS in deionized water (2.9 mg/mL) was added EDC (45 mg) and NHS (11 mg) to a final volume of 3 mL. After stirring for 30 minutes the solution was used for electrode functionalization.

Methods and Materials—Electrode functionalization with NHS-activated AAOT:PSS and PEDOT:PSS: Gold electrode strips were cleaned by successive washes with acetone, isopropanol, and deionized water and after drying, treated with UV-ozone for 15-20 minutes to remove organic contaminants. To completely cover the electrode surface 40 L of NHS-activated AAOT:PSS or PEDOT:PSS at 1-2% w/v were drop casted on the gold electrodes. The PEDOT:PSS coated electrodes were dried at room temperature to enhance the films' electrical conductivity and stability. Without drying, NHS-activated AAOT:PSS on the gold surface was further modified for covalent conjugation of Streptavidin.

Methods and Materials—Covalent immobilization of Streptavidin on NHS-activated AAOT:PSS and PEDOT:PSS: The NHS-activated AAOT:PSS and PEDOT:PSS electrodes were reacted with 20 μL streptavidin (100 g/mL) for 1-2 hours. After washing with phosphate-buffered saline to remove unbound streptavidin, the functionalized electrodes were used for immobilizing the anti-miRNA oligonucleotides.

Methods and Materials—Conjugation of anti-miR to the Streptavidin-immobilized electrode surface: Electrodes were functionalized with oligonucleotides having biotin for immobilization and methylene blue as the electrochemical reporter. A solution of the anti-miRNA oligonucleotide (4 μL, 5 μM) was carefully applied to the streptavidin-modified electrode surface and incubated at room temperature for 3 hours to promote the specific biotin-streptavidin interaction. Attachment and orientation of the oligonucleotides (anti-miRs) were optimized to allow subsequent electrochemical detection, with methylene blue providing a measurable redox signal for monitoring target miRNA hybridization.

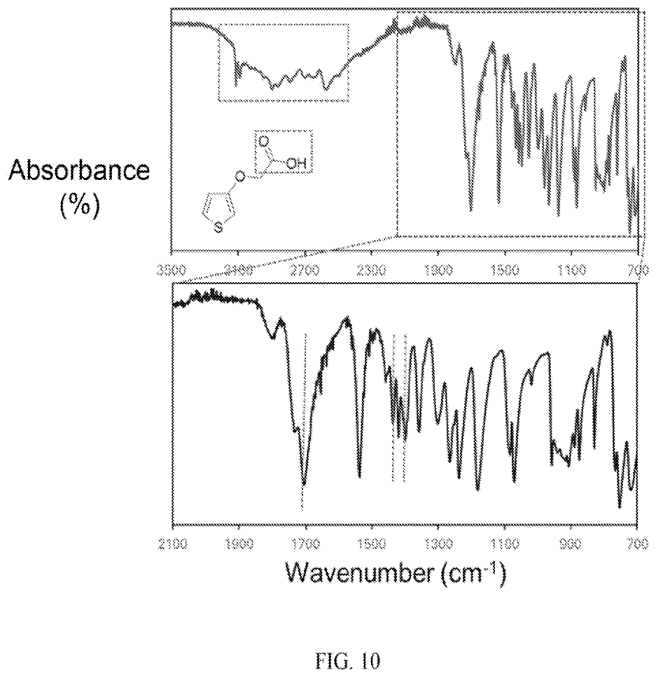

Results—Designing post-synthetically modifiable conductive polymer AAOT:PSS and functionalization of electrodes: The chemical structure of the AAOT monomer was confirmed by HR-MS, ¹H, ¹³C NMR and IR spectroscopy (see experimental and FIGS. 10-12). FTIR spectrum for AAOT (FIG. 10) shows the stretching vibration of C═O near around 2500-3300 and 1700 cm⁻¹ confirming the presence of the carboxylic acid group.

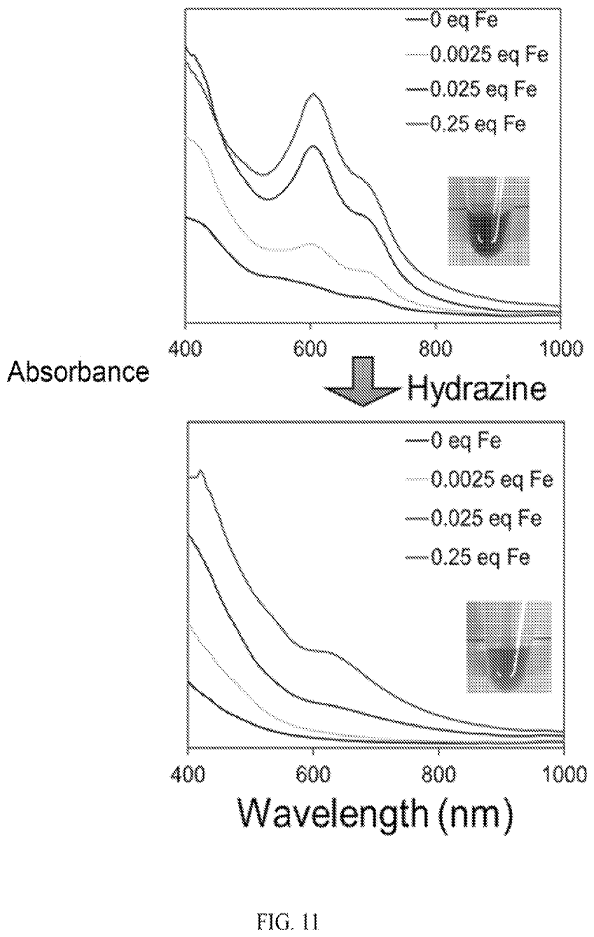

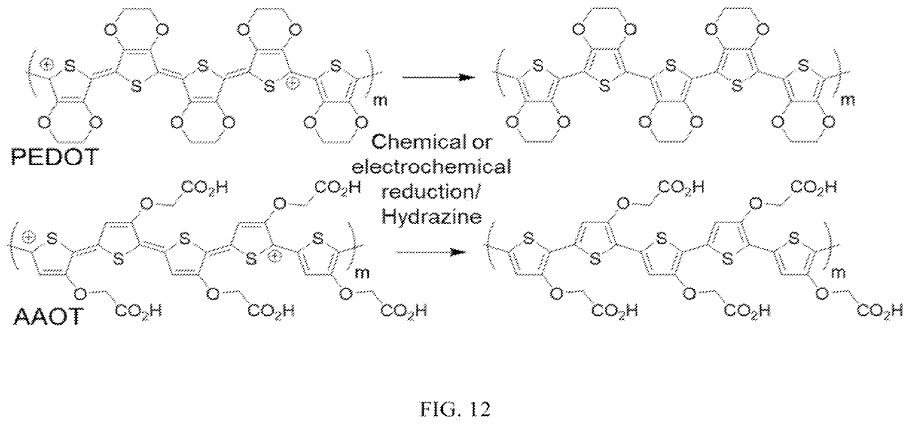

Oxidative polymerization of AAOT in the presence of poly(styrene sulfonic acid) (PSS) using the Fe/(NH₄)₂S₂O₈ initiation system (molar ratio Fe/AAOT=0.25, 0.025 and 0.0025), led to a gradual change from colorless to green indicating that polymerization occurred (FIG. 11). UV-Vis showed an absorption maximum at around 606 nm with a shoulder at around 700 nm (solid line in FIG. 11). PEDOT:PSS has polarons and bipolarons that upon reduction yield a neutral conjugated poly(thiophene) backbone. This so-called dedoping can be done by reducing agents like hydrazine. To see whether such structures are present in our AAOT:PSS system, we reacted the solution with hydrazine and measured UV-Vis. Upon adding hydrazine the signals at 606 nm and 700 nm in the UV-Vis spectrum disappeared and the original green solution turned brown (FIG. 11). The reduction scheme of AAOT in presence of hydrazine has been shown in FIG. 12 which resembles similar reduction process as PEDOT.

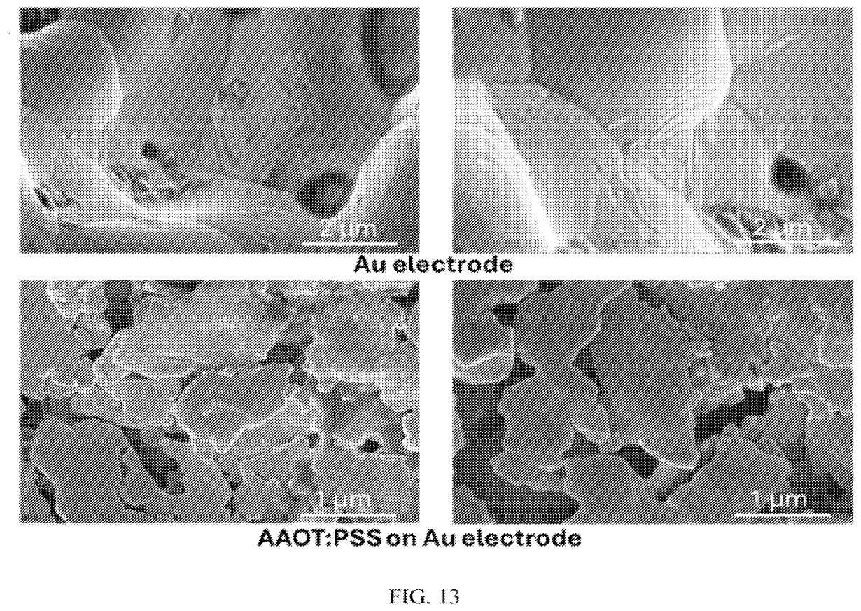

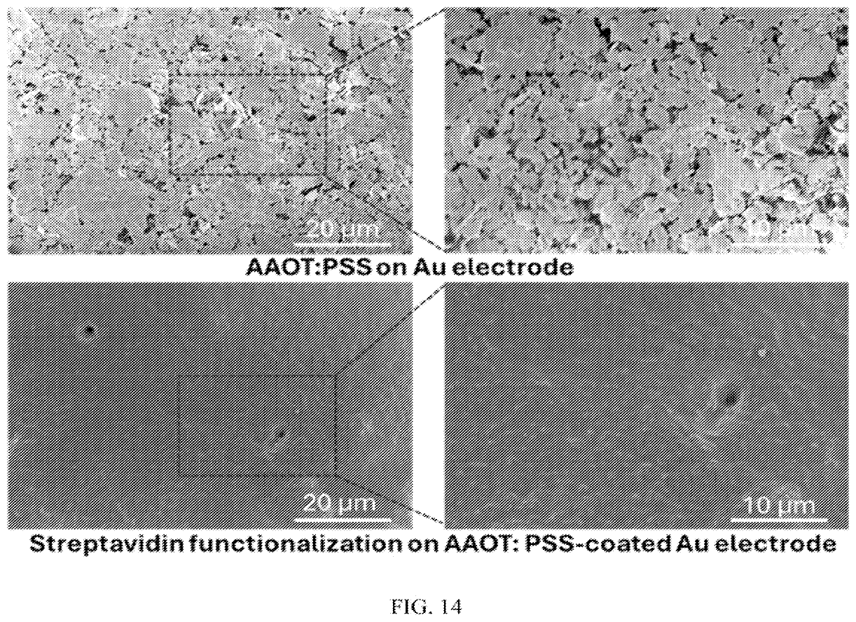

The AAOT:PSS solution after dialysis (molar ratio Fe/AAOT=0.025) showed presence of nanoparticles about 300 nm in diameter. It is important to mention that DLS of PSS solution alone (data not shown) does not show these structures, further supporting the formation of AAOT:PSS nanostructures. At Fe/AAOT=0.0025, the hydrodynamic diameter of the particles remained the same at around 288 nm with PDI values of ˜0.23, confirming a relatively high monodispersed polymer solution. SEM images show different topologies after functionalizing with AAOT:PSS and Streptavidin immobilization (FIGS. 13-14). SEM images were taken after washing with water after each level of functionalization. SEM images confirm successful well-dispersed immobilization of Streptavidin on AAOT-PSS decorated Au electrode surface.

Results—Selection of Target miRNAs for mTBI (Mild Traumatic Brain Injury) and Design of Single-Stranded Oligos: The selection of salivary miRNAs for diagnosing mild traumatic brain injury (mTBI) involves identifying those miRNAs that are relevant to brain injury mechanisms, easily detectable in saliva, and demonstrate high diagnostic accuracy and stability. Among the 31 miRNAs examined, miR-30e, miR-21, and let-7a were chosen based on their biological relevance and specific characteristics. miR-30e (sequence: UGU AAA CAU CCU UGA CUG GAA G) is linked to neuronal apoptosis and cellular stress responses, miR-21 (sequence: UAG CUU AUC AGA CUG AUG UUG A) is involved in regulating inflammation and apoptosis, and let-7a (sequence: GGU AGU AGG UUG UAU AGU U) plays a role in cell differentiation and proliferation. The miRNA and anti-miRNA sequence details are included in Table 1 and Table 2.

TABLE 1

| Anti-miR | Sequence |
|---|---|
| let-7a | MB-AACTATACAACCTAC |
| (miR-let-7a-5p) | TACCTCA-Biotin |
| miR-30e | MB-CTTCCAGTAAGGATG |
| (miR-30e-5p) | TTTACA-Biotin |
| miR-21 | MB-TCAACATCAGTCTGA |
| (miR-21a-5p) | TAAGCTA-Biotin |

TABLE 2

| miRNA | Sequence |
|---|---|
| let-7a | UGA GGU AGU AGG UUG UAU AGU U |

(miR-let-7a-5p)

| | |
|---|---|
| miR-30e | UGU AAA CAU CCU UGA CUG GAA G |
| (miR-30e-5p) | |
| miR-21 | UAG CUU AUC AGA CUG AUG UUG A |
| (miR-21a-5p) | |

Complementary single-stranded oligonucleotides (ASOs) or anti-miRs were designed to detect these miRNAs, ensuring sequence specificity and optimal thermodynamic properties to form stable hybridization between anti-miR and target miRNA at physiological conditions. These ASOs were chemically modified to improve stability and enable host-guest conjugation among biotinylated anti-miRs and streptavidin, enhancing their electrochemical signaling ability. Specifically, biotin and methylene blue modifications were added at opposite ends of the oligos to increase electrochemical and photophysical stability, specificity, and binding affinity through host-guest interactions. Biotin's ability to bind to streptavidin, which is covalently attached to designed conductive polymer on electrodes, facilitates the system's stability. Methylene blue is a redox reporter, producing signals in response to conformational changes or variations in intramolecular distances when hybridization occurs between the ASOs and their target miRNAs. This innovative approach harnesses the stability and diagnostic potential of salivary miRNAs, offering a promising avenue for non-invasive mTBI diagnostics, capitalizing on the growing interest in miRNAs as reliable biomarkers for neurological conditions.

Results—Electrochemical miRNA Detection and comparison between AAOT:PSS and PEDOT:PSS: We employed cyclic voltammetry (CV) and differential pulse voltammetry (DPV) for miRNA detection (FIG. 1), given their well-established use in electrochemical sensing. CV and DPV were conducted for specific miRNAs (miR-30e, miR-21, let-7a) using miR-specific methylene blue (MB)-labeled antisense oligonucleotide (ASO) probes conjugated to designed conductive polymers. As described above, the polymers were post-synthetically modified by streptavidin immobilization for subsequent binding to biotinylated ASOs. Hybridization of single-stranded oligonucleotides, while not altering the system's overall charge, modifies the intramolecular charge distribution. When exposed in the single-stranded state, negatively charged phosphate groups become partially buried within the double helix upon hybridization, increasing electrostatic repulsion between strands. This change in charge distribution and electrostatic interactions is detected via CV, which measures current response changes to varying voltage. The change in electrical output has been demonstrated diagrammatically in FIG. 1. Specifically, oligo hybridization affects guanine base accessibility and electron transfer, leading to CV oxidation peak current and potential shifts. Thus, we indirectly detect and quantify miR and anti-miR oligo hybridization by monitoring CV signal changes associated with guanine oxidation and electron transfer.

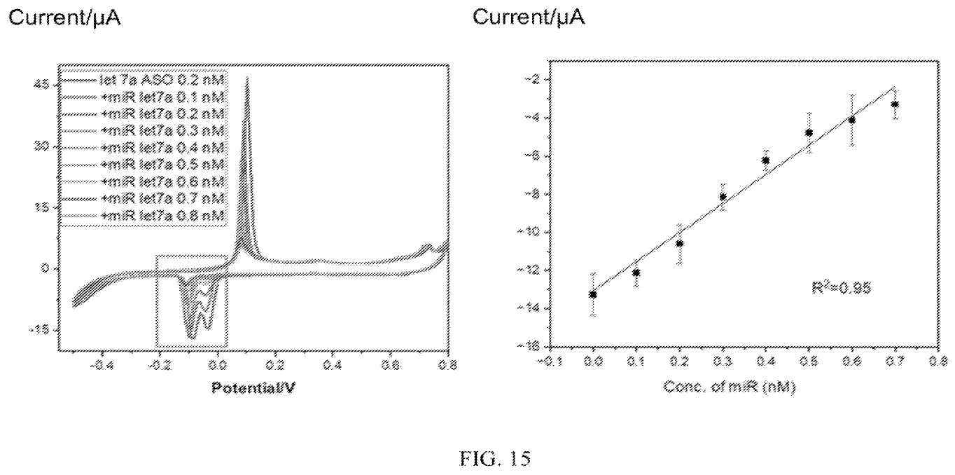

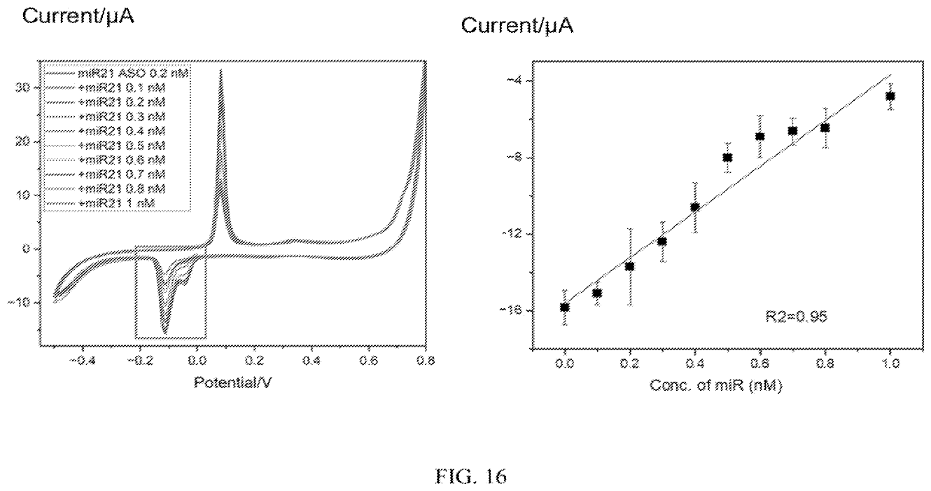

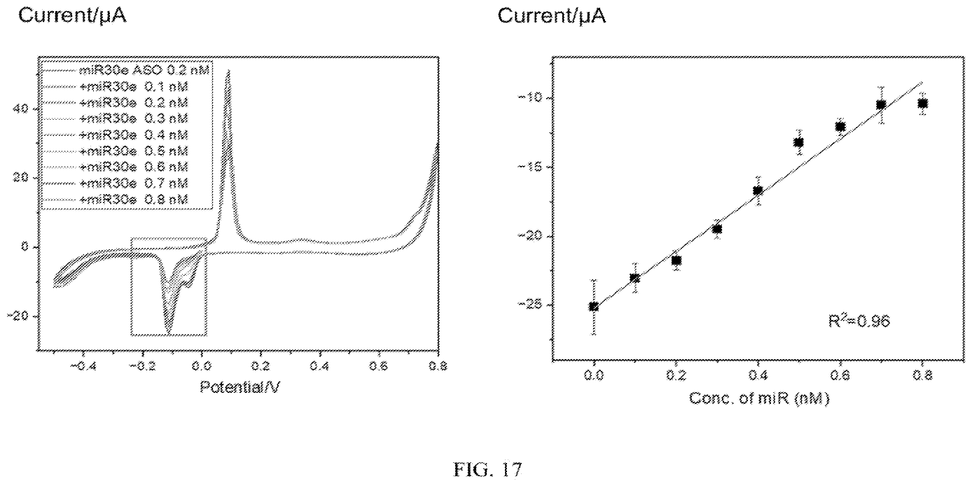

The current output for MB attached to the ASO decreased as MB stretched away from the electrode surface upon binding with the complementary miRNA, reducing electron transfer efficiency (FIGS. 15-17). A gradual decrease in CV current was observed with increasing target miRNA concentrations (FIGS. 15-17), consistently seen for miR-30e, miR-21, and let-7a. For miR-21, a linear current decrease (AI) with increasing miRNA concentrations was observed, with a limit of detection (LOD) between 0.23-0.32 nM and a limit of quantification (LOQ) between 0.7-1 nM. Similar trends were observed for all three miRs, with a slight peak current shift also noted.

DPV spectra didn't show significant changes like CV at very low concentrations. Generally, DPV offers higher sensitivity due to reduced background noise and enhanced signal-to-noise ratio, minimizing capacitive currents and highlighting faradaic processes. In our platform, DPV didn't scale as effectively with increased surface activity from functionalization, making CV more responsive and the main electrochemical technique for our AAOT:PSS functionalized electrodes.

The complex functionalization process enhanced electron transfer kinetics and increased the Au-electrode active surface area. This facilitated stronger miR and MB-miR-Biotin ASO interactions, leading to more pronounced CV redox signals. Due to superior CV signals over DPV at low concentrations, further electrochemical assessments with mTBI patients' saliva samples were carried out with CV only. The comparison between traditional PEDOT:PSS functionalized Au electrodes and the AAOT:PSS functionalized Au electrodes for all the three miRNAs were carried out using cyclic voltammetry.

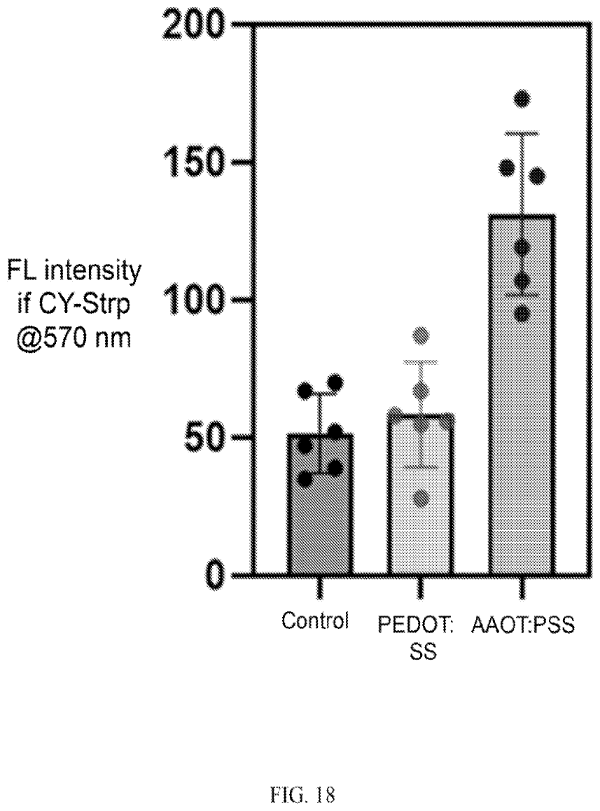

To check distribution and immobilization of streptavidin over the polymer (PEDOT and AAOT) functionalized electrodes, commercially available fluorescent streptavidin (Strp-Cy3) was used and conjugated over the polymer-fabricated electrodes in the same process as streptavidin. The fluorescence intensity in FIG. 18 shows the difference between covalently conjugated Streptavidin-Cy3 on AAOT and loosely attached Streptavidin-Cy3 on PEDOT after a single wash. The covalently conjugated Streptavidin-Cy3 on AAOT retained significantly higher fluorescence intensity, indicating a more stable and robust attachment. In contrast, the loosely attached Streptavidin-Cy3 on PEDOT exhibited a pronounced loss of fluorescence, emphasizing the superior covalent binding stability achieved with AAOT.

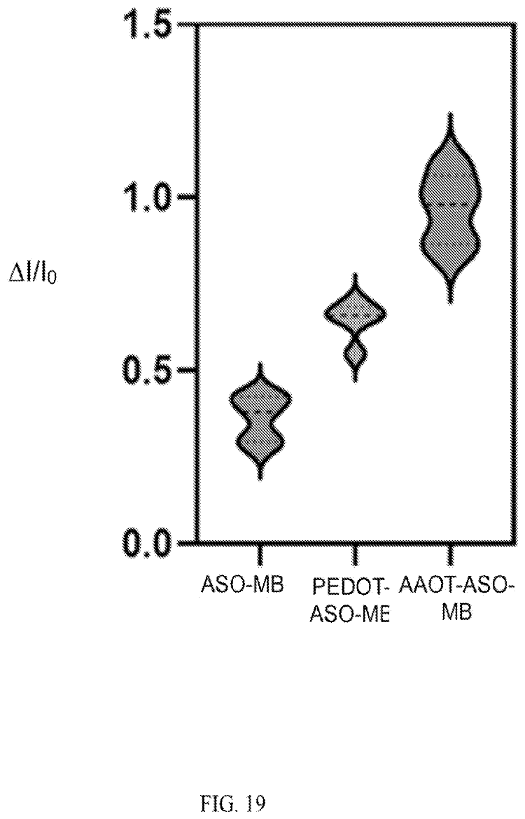

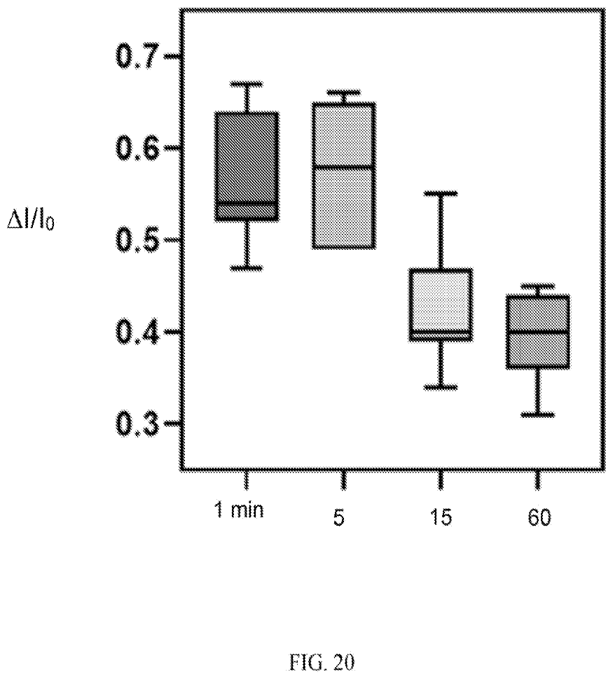

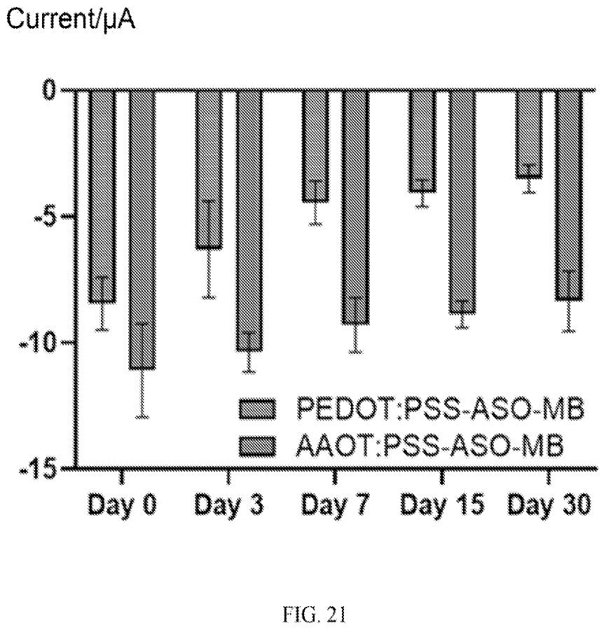

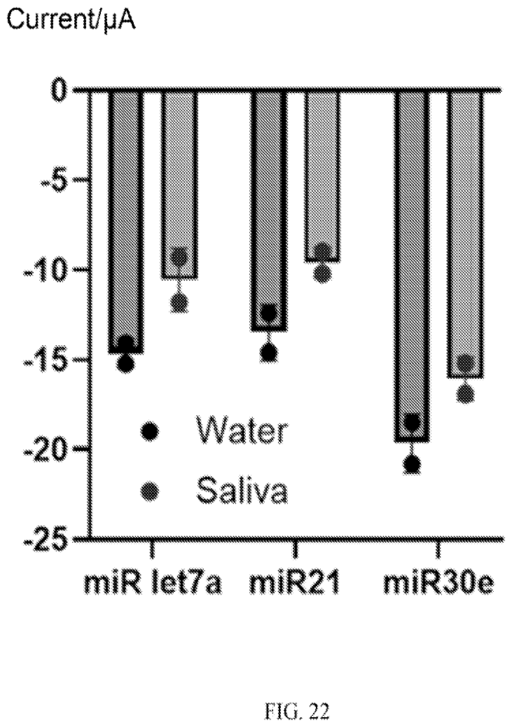

Current output of electrodes functionalized with AAOT increased markedly upon functionalization. This enhancement suggests effective integration of the functional groups, leading to improved electron transfer capabilities (FIG. 19). The consistent current output of AAOT electrodes over time, illustrated in FIG. 20, further underscores the reliability of this material. Even 60 minutes after hybridization between anti-miR and miRNAs, the current output remained stable, showcasing the material's capability for sustained signal detection. FIG. 21 presents the long-term stability of the PEDOT and AAOT functionalized electrodes. AAOT and PEDOT both demonstrated stable performance over 30 days, but AAOT exhibited a superior retention of electrochemical activity. This long-term stability is crucial for practical sensor applications, where consistent performance over extended periods is essential. Minimal matrix effect was observed in saliva during CV measurements (FIG. 22). miRNAs were spiked in artificial saliva to check matrix effect, and it did not significantly interfere with the signal, indicating that the sensor's performance remains reliable in complex biological fluids. This finding suggests that the developed AAOT:PSS functionalized Au electrodes are well-suited for non-invasive, real-time detection of miRNAs in saliva, a critical factor for the diagnosis of mTBI related salivary miRNAs.

Overall, the AAOT:PSS outperforms PEDOT:PSS in terms of binding stability, current output, and long-term reliability, making it a more suitable candidate for developing sensitive and stable electrochemical biosensors for the detection of salivary miRNAs.

Results—Detection of miRNAs from mTBI patients' saliva Samples: Picomolar quantities of miRNA were successfully detected in saliva samples from 14 patients with mild traumatic brain injury (mTBI) and shown in Table 3. The miRNA was extracted from the saliva through the standard miRNA extraction kit, where the miRNA concentration in saliva was concentrated 10 folds, which was accounted for while performing the calculations.

TABLE 3

| Patient ID | miR 1et7a (nM) | miR 21 (nM) | miR 30e (nM) |
|---|---|---|---|
| PSU. Sample 1 | 0.14 | 0.16 | 0.2 |
| PSU. Sample 2 | NA | NA | NA |
| PSU. Sample 3 | 0.21 | 0.2 | 0.14 |
| PSU. Sample 4 | 0.17 | 0.14 | 0.17 |
| PSU. Sample 5 | 0.17 | 0.17 | NA |
| PSU. Sample 6 | NA | 83 | 0.10 |
| PSU. Sample 7 | 0.22 | 0.19 | 0.17 |
| PSU. Sample 8 | 0.19 | 0.2 | 0.20 |
| PSU. Sample 9 | 0.15 | 0.14 | 0.16 |
| PSU. Sample 10 | 0.82 | 0.74 | NA |
| PSU. Sample 11 | NA | 0.19 | 0.16 |
| PSU. Sample 12 | 0.2 | 0.18 | 0.17 |
| PSU. Sample 13 | 0.19 | 0.14 | 0.13 |
| PSU. Sample 14 | 0.17 | 0.13 | 0.13 |

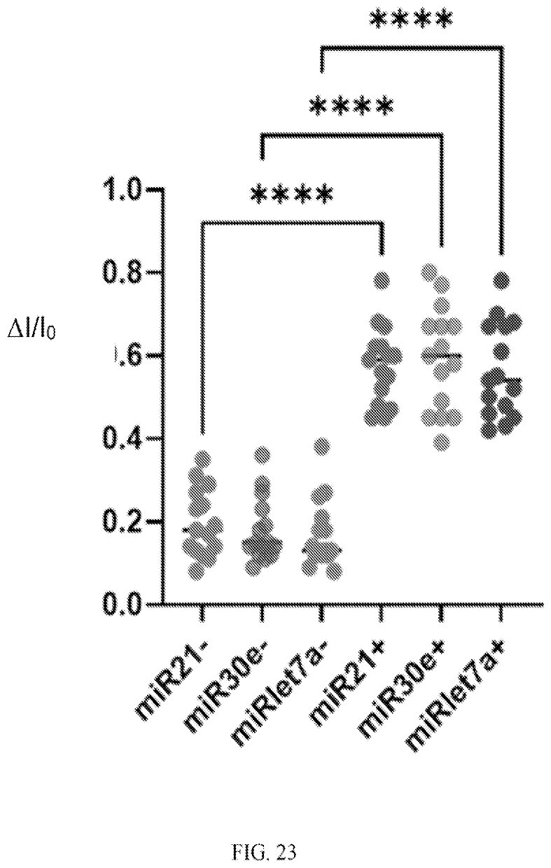

The miRNA concentrations were measured using cyclic voltammetry, and the values were determined from previously established standard curves for the corresponding miRNAs (FIGS. 15-17). A two-way ANOVA was conducted on the grouped clinical samples (positive and negative for all three miRNAs), revealing a highly significant correlation between control and positive samples (****, p<0.0001), confirming the sensor's accuracy (FIG. 23).

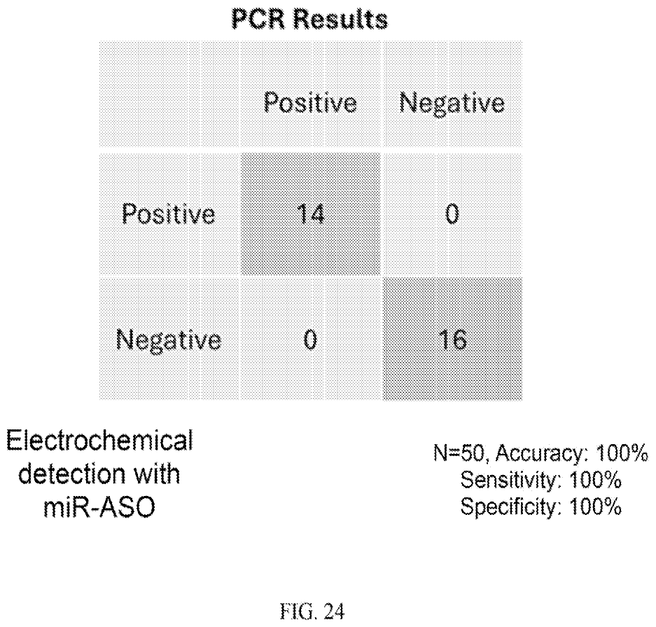

Significant differences in miRNA levels were observed in miR let7a, miR21, and miR30e positive samples when measured using cyclic voltammetry, with results expressed as ΔI/I₀, where I₀ represents the initial current output before sample addition, and ΔI is the change in current (I) following sample introduction. The sensor demonstrated 100% sensitivity and specificity, accurately evaluating all 30 patient samples (14 positive). The confusion matrix (FIG. 24) illustrates the accuracy of the electrochemical detection and its correlation with PCR results.

### Example 2: COF-Based Conductive Electrode and Electrochemical Sensor

Methods and Materials—Synthesis: 1,3,5 triphenylphloroglucinol was synthesized using the following method. In a reaction vessel, hexamethylenetetraamine (15.10 g) and dried phloroglucinol (23.72 g) were combined under an inert nitrogen atmosphere. Trifluoroacetic acid (90 mL) was added, and the mixture was heated to 100° C. for approximately 2.5 hours. Subsequently, 150 mL of 3 M hydrochloric acid was introduced, and heating was maintained at 100° C. for an additional hour. After cooling to ambient temperature, the solution was filtered through Celite and extracted with dichloromethane (ca. 350 mL). The organic layer was dried over magnesium sulfate, filtered, and concentrated under reduced pressure to yield a proportionate amount of an off-white powder. The product exhibited high purity as determined by ¹H NMR and could be further purified by sublimation if desired.

Methods and Materials—Powder X-ray diffraction: Powder X-ray diffraction (PXRD) was conducted using a Rigaku MiniFlex 600, scanning at a rate of 10°/min within the 2θ range of 3 to 40°, utilizing graphite monochromatized Cu Kα radiation (λ=0.15405 nm).

Methods and Materials—Scanning Electron Microscopy: Sample morphology was examined using a field-emission scanning electron microscope (FE-SEM, S-4800, Hitachi) with an energy-dispersive X-ray (EDX) spectrometer. The COF and COF-polymer-based platforms were drop casted on the Au-electrode, and the sensor strip containing the electrode was imaged before and after the addition of each layer and the electrodes are fixed on SEM stubs coated with double-sided carbon tape, air-dried, and then sputter coated with 80% platinum/20% palladium to achieve conductivity. The FEI Nova NanoSEM 450 was then used to monitor the surface topography of each of the samples. The field-emission scanning electron microscopic images were recorded. The respective EDS spectra were also recorded for each of the samples.

Methods and Materials—Raman Spectroscopy: Raman spectra were collected using a Renishaw inVia Reflex Raman Spectroscope system with the following parameters: 785 nm laser, 45 mW (50%) power, grating of 1200, 100× magnification, acquisition time 0.3 s with the center of Raman frequency set at 1100 cm¹.

Methods and Materials—X-ray Photoelectron Spectroscopy: The samples were drop-casted and vacuum-dried each time to form a thick layer. XPS experiments used a Physical Electronics VersaProbe III instrument with a monochromatic Al kα X-ray source (hv=1486.6 eV) and a concentric hemispherical analyzer. Charge neutralization was performed using low-energy electrons (<5 eV) and argon ions. The binding energy axis was calibrated using sputter-cleaned Cu (Cu 2p3/2=932.62 eV, Cu 3p3/2=75.1 eV) and Au foils (Au 4f7/2=83.96 eV). Peaks were charge referenced to the CHx band in the carbon is spectra at 284.8 eV. Measurements were made at a takeoff angle of 450 concerning the sample surface plane. This resulted in a typical sampling depth of 3-6 nm (95% of the signal originated from this depth or shallower). Quantification was done using instrumental relative sensitivity factors (RSFs) that account for the X-ray cross-section and inelastic mean free path of the electrons. The analysis size was ˜200 μm in diameter. The analysis was performed in CasaXPS software.

Methods and Materials—NMR Spectroscopy: NMR experiments were performed on a Bruker Avance II 300 MHz spectrometer (7.05T, 11B: 96.38 MHz). Single-pulse excitation with a short rf pulse of 1 us was used to acquire the spectra. 4096 scans were collected with a relaxation delay of 4 s. All spectra were recorded at ambient temperature with 12 kHz magic angle spinning. The chemical shift was referenced to (δ=0 ppm). Spectral line shape fits were calculated using the solid line shape analysis (SOLA) program in Topspin. The fitting model included chemical shift anisotropy and quadrupolar interaction for 4 different chemical sites. Topspin software was used for analyzing the data.

Methods and Materials—Thermogravimetric Analysis: Thermogravimetric analysis (TGA) data were collected with a Thermal Analysis Instrument (SDT 2960, TA Instruments, New Castle, DE), operating at a heating rate of 10°/min under a nitrogen flow of 100 mL/min.

Methods and Materials—Fluorescence Microscopy: Images were obtained with a Zeiss LSM 880 confocal microscope. Fluorescence imaging of Cy3-streptavidin was performed using Cy3 specific fluorescent channel.

Methods and Materials—UV-vis and Fluorescence Spectroscopy: UV-vis absorption spectra were measured with a Hitachi U-3100 spectrophotometer. The total miR concentration was determined using Thermo Scientific™ NanoDrop™ OneC Microvolume UV-vis Spectrophotometer. The absorbance spectra for the assay with 96-well plates were recorded on Biotek Synergy Neo2Microplate Reader for endpoint, kinetic, and spectral analyses. Each experiment was repeated at least three times and an average of these spectra were presented. Two samples from each category were selected randomly to standardize the assays. The absorbance spectra were then normalized. The highest absorbance value was chosen for each spectrum, and then we divided the absorbance values by that number. The normalized data was then compared to standardize the assay parameters, regardless of the details of the experiment. Fluorescence spectra were recorded in the solid and solution phases in the Horiba Fluromax plus fluorescence spectrophotometer.

Methods and Materials—FTIR Measurements: Fourier transform infrared (FTIR) spectra were recorded using a Vertex PerkinElmer 580BIR spectrophotometer (Bruker) employing the KBr pellet technique.

Methods and Materials—Transmission Electron Miscroscopy: Transmission electron microscopy (TEM) images were taken on an FEI Tecnai G2 S-Twin equipped with a field emission gun operating at 200 kV. 20 μL solution of all the samples was added on top of a carbon-coated copper grid (400 mesh). This was allowed to stay for ˜10 min before being removed with a filter paper and imaged under a transmission electron microscope (FEI tecnai T12). The tungsten filament with 80 kV accelerating voltage was used for the investigations.

Methods and Materials—Electrochemical Measurements: The electrochemical measurements were performed using the electrochemical sensor interface PalmSens Sensit-smart (Palm Instruments, The Netherlands), controlled by a PC running PSTrace software version 5.6 and mobile based android app PSTouch. The three-electrode Zimmer & Peacock electrochemical cell (L4.5×W0.8 cm) has a gold working electrode. Each electrode is produced by screen-printing technology and constitutes of a rectangular gold working electrode (3 mm diameter), a silver pseudoreference electrode and a graphite counter electrode.

The difference in charge transfer capacitance (electrochemical potential difference) of COF-polymer-ASO functionalized electrodes upon interaction with target miRNAs was measured by the portable device-based biosensor. The DPV and CV parameters were optimized. CV data were recorded using a current range of 100 μA to 10 mA with a potential range of −0.7 V to 0.7 V having step of 0.1 V and 3 number of scans were taken. For the DPV experiments the current range was selected from 1 μA to 10 mA. Potential range was set from −0.5 V to 0.5 V with a step of 0.01 V. Then, miRNAs were gradually added to it at different concentrations, and CV, DPV, and graphs were generated as standard curves. mTBI salivary samples were isolated through miRNA extraction and then drop-cast over the functionalized electrodes.

Results—Selection of Target miRNAs for mTBI (Mild Traumatic Brain Injury) and Design of Single-Stranded anti-miRNA Oligos: The selection of target miRNAs for mTBI in human saliva involves identifying miRNAs that are pathophysiologically relevant to brain injury, detectable in saliva and exhibit high diagnostic accuracy and stability. Among the 31 miRNAs analyzed, miR-30e, miR-21, and let-7a were selected based on specific criteria. miR-30e (sequence: UGU AAA CAU CCU UGA CUG GAA G) is involved in neuronal apoptosis and stress responses, miR-21 (sequence: UAG CUU AUC AGA CUG AUG UUG A) plays a role in inflammation and apoptosis regulation and let-7a (sequence: GGU AGU AGG UUG UAU AGU U) regulates cell differentiation and proliferation.[36] Designing complementary single-stranded oligonucleotides (ASOs) or anti-miRNAs (anti-miRs) against these miRNAs involves ensuring sequence specificity and optimal thermodynamic stability. The anti-sense oligos were designed to incorporate chemical modifications for host-guest conjugation and generate electrochemical signals upon hybridization with complimentary miRNAs (FIG. 2). The oligos were fabricated with methylene blue and biotin modifications at their opposite ends to enhance electrochemical and photophysical specificity, stability, and host-guest-based binding affinity. This approach leverages miRNAs' stability and diagnostic potential in saliva to develop non-invasive tools for mTBI diagnosis. Biotin can bind to Streptavidin, which can be immobilized on the electrodes, and methylene blue acts as a redox reporter upon conformational or intramolecular distance-dependent changes. The details of miRNA sequences and anti-miR oligonucleotide sequences are provided in Table 1 and Table 2 (provided in Example 1).

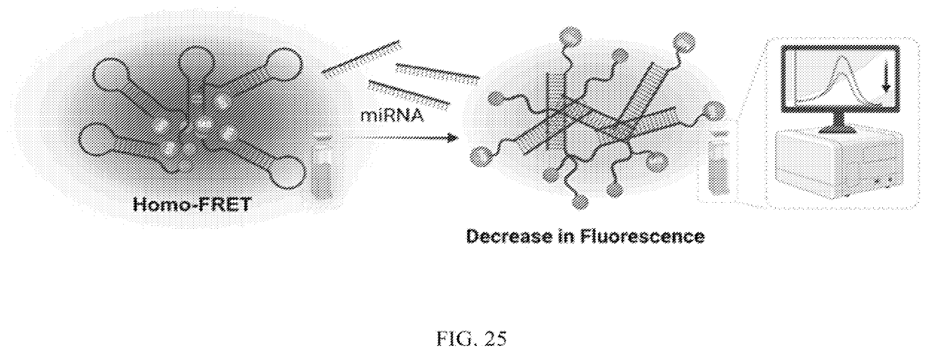

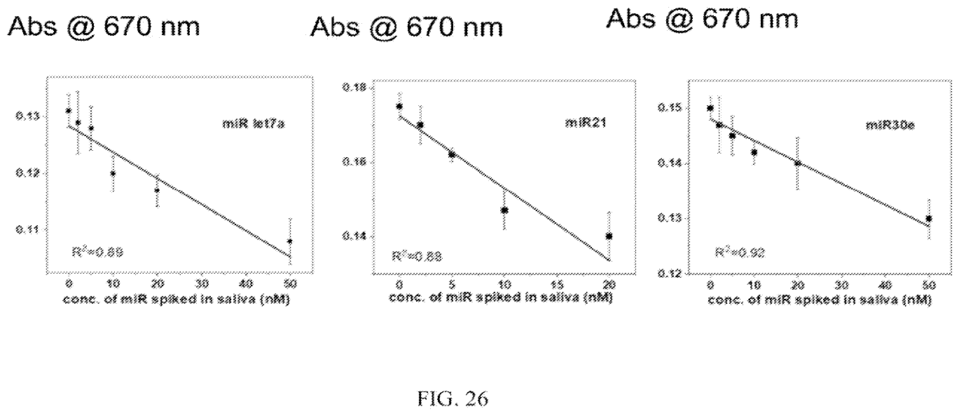

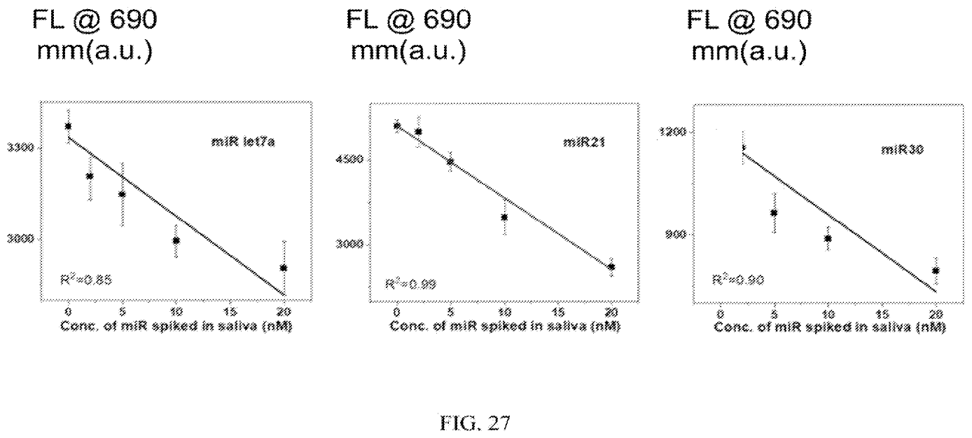

Results—Validation and Specificity of anti-miRNA Oligos with Spectroscopy (UV-Vis and Fluorescence): The specificity of MB-tagged ASOs against their corresponding miRNAs was validated by spectroscopic measurements performed in aqueous medium and artificial saliva. The quenching of fluorescence has been explained schematically in FIG. 25. Artificial saliva was used to evaluate its impact on photophysical measurements, revealing minimal effects in both solution-phase cuvettes and electrochemical detection on electrodes. A gradual decrease in methylene blue (MB) absorbance (670 nm) and fluorescence (690 nm) was observed during hybridization with increasing concentrations of miRNAs (miR let-7a, miR-21, miR-30e) in artificial saliva. This decrease is attributed to the conformational changes in MB-tagged antisense oligonucleotides (ASOs) upon binding with complementary miRNAs, altering the MB molecules' electronic environment. The conformational change brings MB closer to the miRNA, facilitating quenching interactions that reduce absorbance at 670 nm (FIG. 26). Hybridization also alters the local environment, including polarity and viscosity, around MB molecules, affecting electronic transitions and further decreasing absorbance. Hybridization induces both static and dynamic quenching of MB fluorescence. Static quenching arises from non-fluorescent complexes formed between MB and the miRNA-ASO hybrid, while dynamic quenching involves increased non-radiative decay due to MB's closer proximity within the hybrid structure. Additionally, Forster Resonance Energy Transfer (FRET) may contribute, as MB's excited-state energy is transferred to nearby MB molecules, with fluorescence intensity affected by differential distances in the hybrid complex (FIG. 25). A concentration-dependent decrease in absorbance and fluorescence was observed in artificial saliva and aqueous media (FIGS. 26-27). This phenomenon highlights the potential of MB-tagged anti-miR ASOs for electrochemical detection, as described later. Raman spectroscopy confirmed successful ASO-miR hybridization in solution and on Au electrodes. Anti-miR and miR interactions on the Au electrode showed changes in surface-enhanced plasmon response, as the hairpin miR-ASO structure stretched upon hybridization with complementary miR, validating the hybridization process.

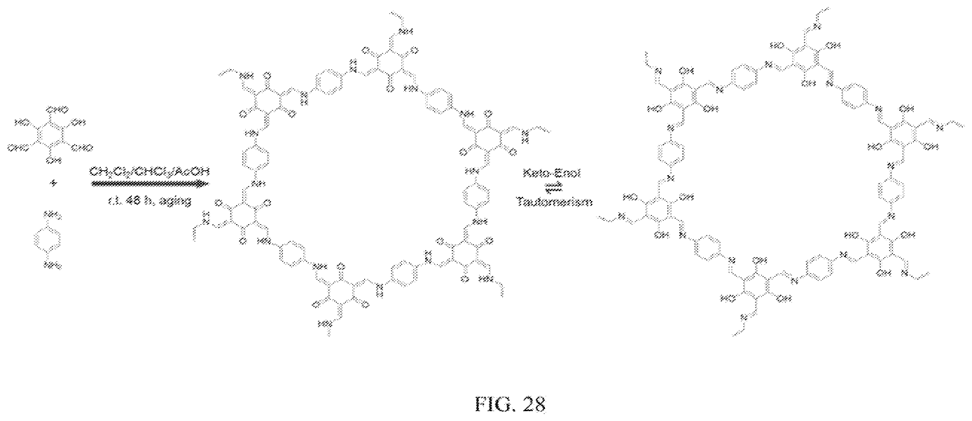

Results—Synthesis of COF and Characterization: 1,3,5-Triformylphloroglucinol was synthesized as described above. 1,3,5-Triformylphloroglucinol was dissolved in a mixture of dichloromethane (CH₂Cl₂), chloroform (CHCl₃), and acetic acid. The resulting homogeneous solution was maintained at room temperature for 48 hours, yielding nanosheet-like covalent organic frameworks (COFs) at the nanoscale level. The choice of the solvent system (CH₂Cl₂ and CHCl₃) was critical in achieving the desired morphology. These solvents ensured the complete dissolution of triformylphloroglucinol, facilitating a slow polymerization, nucleation, and growth process that favored the formation of multilayer COF sheets and in contrast, other solvents partially dissolved triformylphloroglucinol, leading to irregular or aggregated particles after the Schiff base reaction. Free hydroxyl groups were required for further functionalization of the COF nanosheets for covalently attaching epoxysilane to attach streptavidin on the COF surface. To promote keto-enol tautomerism in these COF nanosheets, a few changes were performed regarding solvent, pH, and temperature (FIG. 28). At slightly lower temperatures, COF nanosheets were dispersed in water containing 5% dimethylformamide, and a minimal amount of NaOH was added to make the solvent slightly alkaline. As the keto form is more stable in higher temperatures, lower pH, and nonpolar solvents, reversing these conditions induces an enol form of COF with flanking hydroxyl groups for further conjugation (FIG. 28).

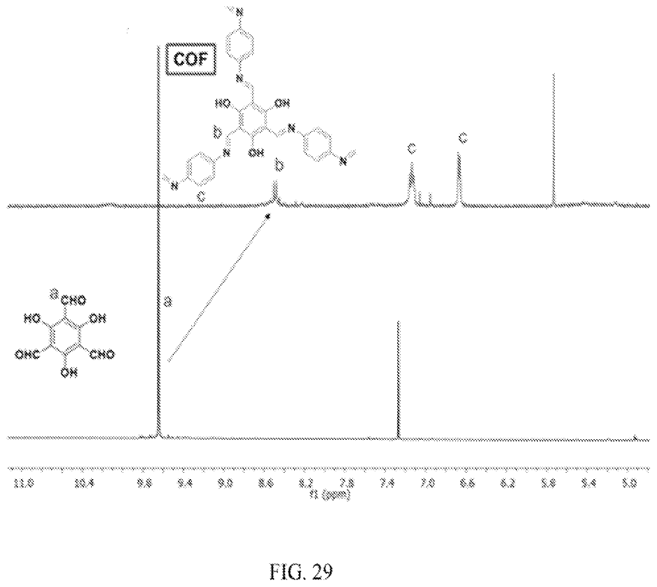

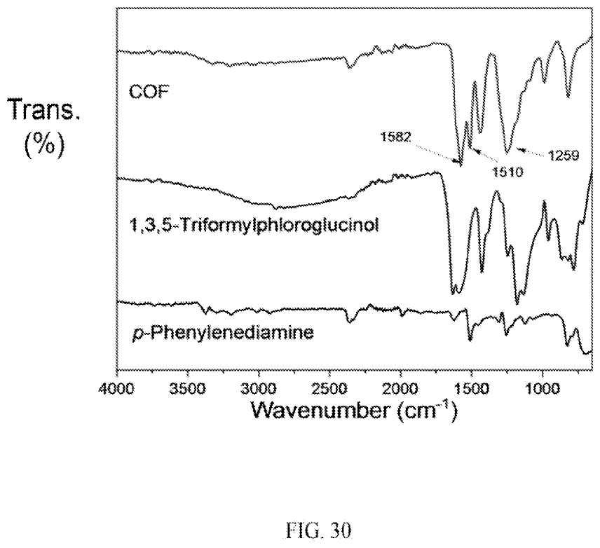

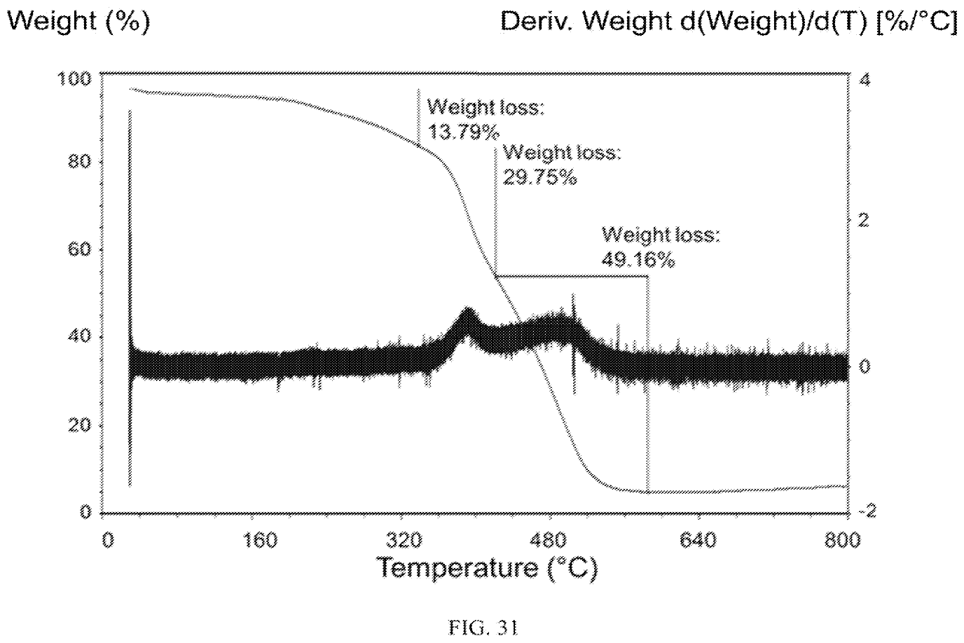

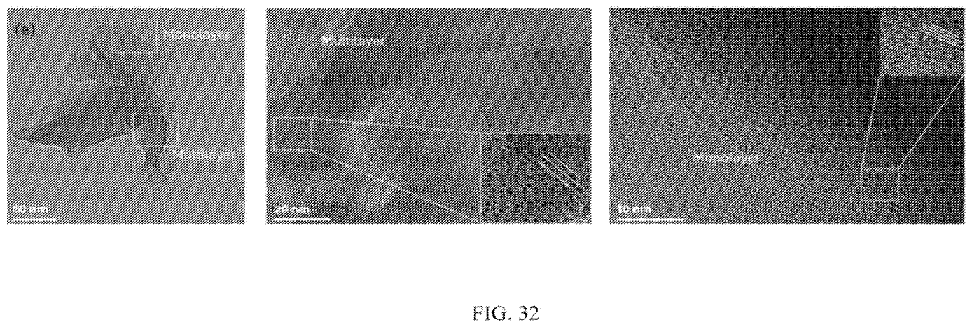

The ¹H NMR spectra in FIG. 29 depict the enol form of the covalent organic framework. In phloroglucinol, the singlet aldehyde proton peak comes within 9-10 ppm, which is visible in NMR. After forming the enol form of COF, proton peaks near 6.6 ppm, and 8.7 ppm denotes the hydroxyl and other benzene ring protons where the aldehyde proton is entirely absent. Aldehyde protons appeared as singlets because there was no neighboring proton with which to couple, but the broadening of peaks in enol-COF was observed due to hydrogen bonding and proton exchange processes. An XRD showed the COF's crystalline nature. Sharp peaks in the lower 20 range (2-10 degrees) correspond to the (100), (110), (200) planes of the nano-framework structure. The broader peaks correspond to smaller repeat distances within the COF structure and represent the stacking of layers or in the nanosheet structure. The XPS analysis of COF provided information on the C1s, N1s, and O1s components. High-resolution XPS analysis provided details on the bonding information of the nano-framework, similar to previous literature.[79] For the C1s spectra, the peak at 284.6 eV represents C—H bonds (backbone), 286.02 eV corresponds to C—N bonds, and 288.3 eV to C=O bonds. In the N1s spectra, peaks at 399.4 eV and 400.1 eV indicate C=N and C—N bonds (imine tautomerism), with a π-π satellite peak at 403.6 eV. The O1s spectra show peaks at 530.7 eV for C=0, 532.6 eV for C—O, and 535.7 eV for O—H, consistent with the COF framework structure. In FIG. 30, the typical C—N and C═C vibrations appeared at 1259 and 1582 cm⁻¹, respectively, in the Fourier transform infrared (FT-IR) spectra, indicating the formation of a b-ketoenamine-linked structure. The FTIR peak at 1510 cm⁻¹ in p-phenylenediamine corresponds to C═C stretching vibrations in the aromatic ring. This characteristic vibration, observed in the 1450-1600 cm⁻¹ range, is influenced by para-positioned amine groups (—NH₂), confirming the benzene ring's presence in the compound. In FIG. 31, thermogravimetric analysis (TGA) shows the COF's high thermal stability, with gradual weight loss from 320° C. to 400° C. and rapid decomposition in two steps between 400° C. and 480° C. While high thermal stability is unnecessary for biochemical sensing, the decomposition profile aligns with regular crystalline COFs, confirming successful synthesis. Changes in thermal stability, as seen in the TGA profile, can also indicate successful modification of the COF nanosheets. The TEM images exhibited the mono and multilayer nanosheet structures with visible lattice fringes for both unmodified and silane-modified COF nanosheets (FIG. 32). Enol-COF was imaged using TEM after dispersing it in a slightly basic aqueous solution and drop-casting onto grids. The images revealed single and overlapping nanosheets (50-200 nm) with a nanoflake-like structure, suitable for coating Au electrodes and providing a larger surface area for modification.

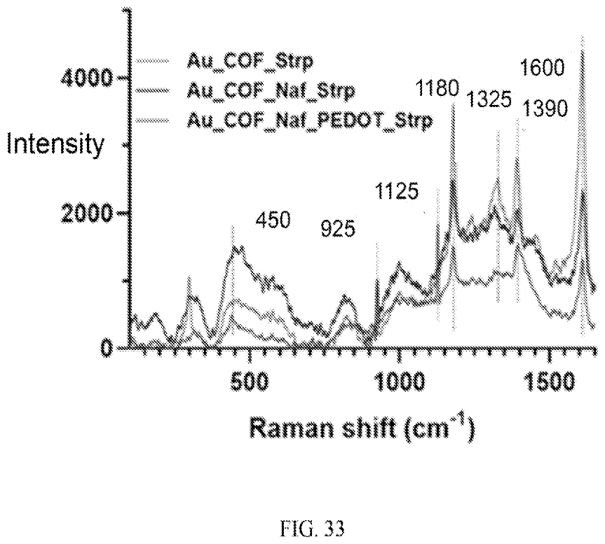

Results—Characterization and Standardization of Post-synthetic Modification of COF Over Electrode Surface for Conjugation of Anti-miRNA Oligonucleotides: The COF was initially characterized using XPS, XRD, NMR, and FTIR spectroscopy, confirming successful fabrication. FTIR, TGA, SEM, and Raman spectroscopy were used to characterize the electrode surface layers after each fabrication step, confirming successful deposition and functionalization. In the FTIR spectra, the emergence of a new peak at 1080 cm⁻¹ indicates the formation of a C—O bond from silane modification. The disappearance of —OH vibrations around 2700-3300 cm⁻¹ after GPTMS functionalization confirms the successful modification of flanking —OH groups as they are replaced by GPTMS. TGA analysis comparing unmodified (blue curve) and modified (green curve) COFs reveals distinct thermal behavior. Both COFs decompose around 100° C., with the modified COF showing earlier, gradual weight loss, indicating potential changes in stability or volatile components from modification. Between 160° C. and 400° C., the modified COF exhibits gradual weight loss, suggesting enhanced thermal stability. At ˜500° C., both COFs decompose significantly, but the modified COF retains more weight, indicating improved thermal stability. SEM shows distinct surface topologies: plate-like COF surfaces, fibrous polymer traces, and bumpy membrane-like protein structures. Raman spectra (FIG. 33) show shifts in key peaks: 450 cm⁻¹ (COF-gold interaction), 925 cm⁻¹ (SO₃⁻ stretching of Nafion), 1125 cm⁻¹ (C—O—C in PEDOT:PSS), 1325 cm⁻¹ (C—N or C═C stretching), 1390 cm⁻¹ (C—H or C—N), and 1600 cm⁻¹ (C═C stretching). Phenylenediamine and 1,3,5-triformylphloroglucinol-based COF were modified with (3-glycidoxypropyl)trimethoxysilane for streptavidin immobilization. The fabrication on carbon and gold electrodes favored Au-electrodes due to better electrical outputs for a portable potentiostat.

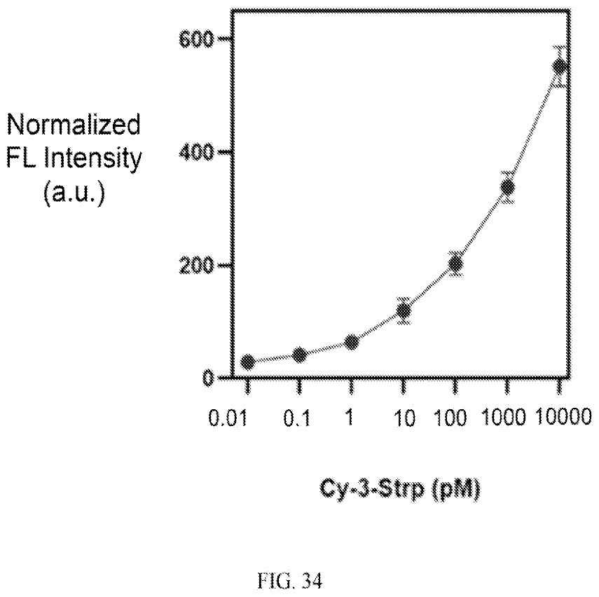

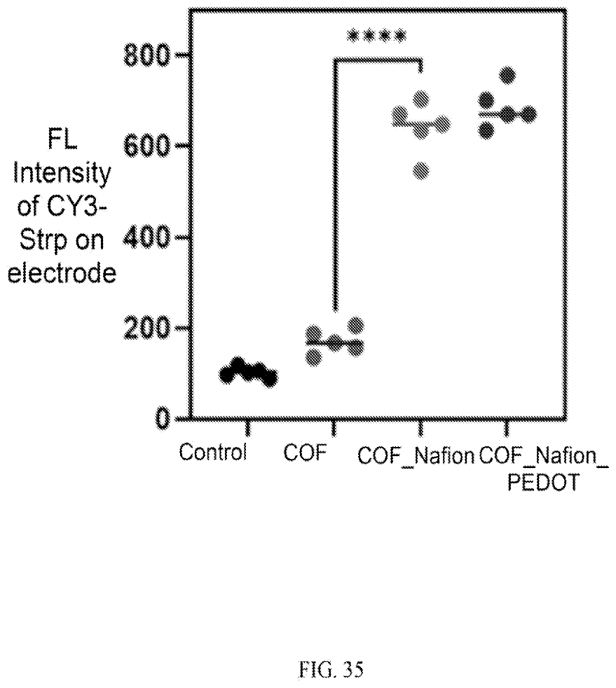

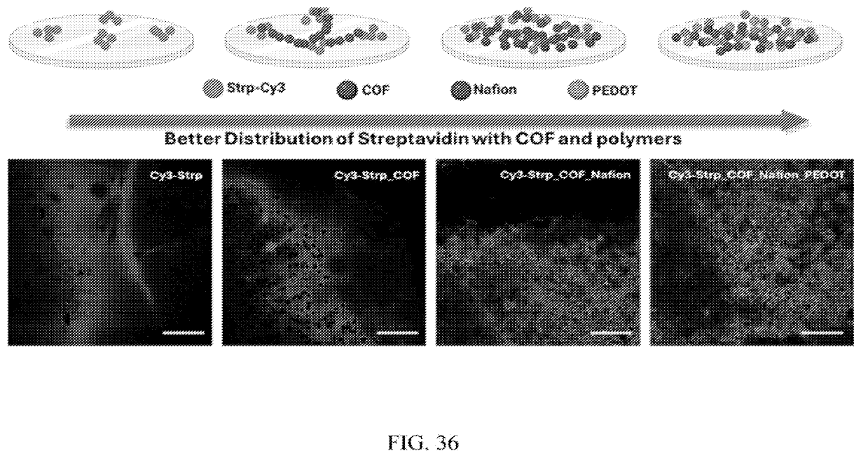

To confirm successful streptavidin conjugation to the COF surface, fluorescent Cy3-streptavidin (Cy3-Strp) was applied to freshly prepared epoxy-functionalized COF-Au electrodes. Using a solid-state sample holder and adjusting it to a 60θ angle towards the detector in a Horiba Fluromax fluorescence spectrophotometer, the fluorescence signal from the electrodes was measured to correlate with fluorophore density. A standard curve was generated by varying Cy3-Strp concentrations, determining 20 g/mL as optimal (FIG. 34). Unmodified electrodes showed fluorescence below detection limits, while epoxy-labeled COF surfaces demonstrated clear Cy3-Strp deposition, indicating effective conjugation (FIG. 35). This density was significantly higher than bare Au electrodes. Confocal microscopy (FIG. 36) revealed that additional Nafion and PEDOT:PSS layers improved Cy3-Strp distribution over the COF-Au surface, as shown schematically in FIG. 36.

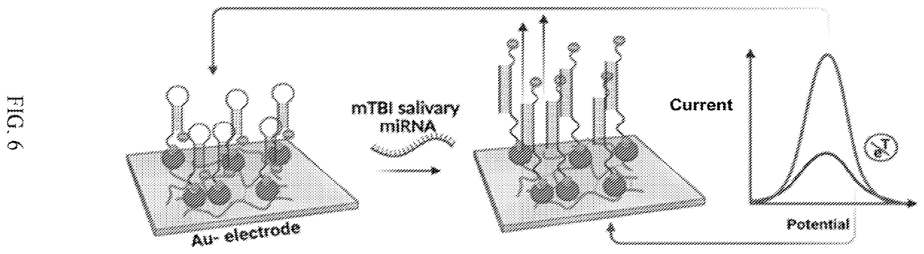

Results—Electrochemical Detection of miRNAs via Cyclic Voltammetry and Differential Pulse Voltammetry: We demonstrated the use of cyclic voltammetry (CV) and differential pulse voltammetry (DPV) for the detection process (FIG. 6), as both techniques are well-studied for electrochemical detection. CV and DPV were conducted for specific miRNAs (miR-30e, miR-21, let-7a) using MB-labeled antisense oligonucleotide (ASO) probes conjugated to phloroglucinol-based COFs. The COFs were modified with GPTMS to introduce epoxy groups for streptavidin immobilization, enabling binding to biotinylated ASOs. Electrochemical parameters are detailed in the experimental section, and the fabrication process was described previously. Hybridization of single-stranded oligonucleotides does not change the overall charge but alters charge distribution. Negatively charged phosphate groups, exposed in single strands, become partially buried in the double helix, increasing electrostatic repulsion. This change affects guanine base accessibility and electron transfer properties, detectable by cyclic voltammetry (CV) as variations in oxidation peak current and potential.

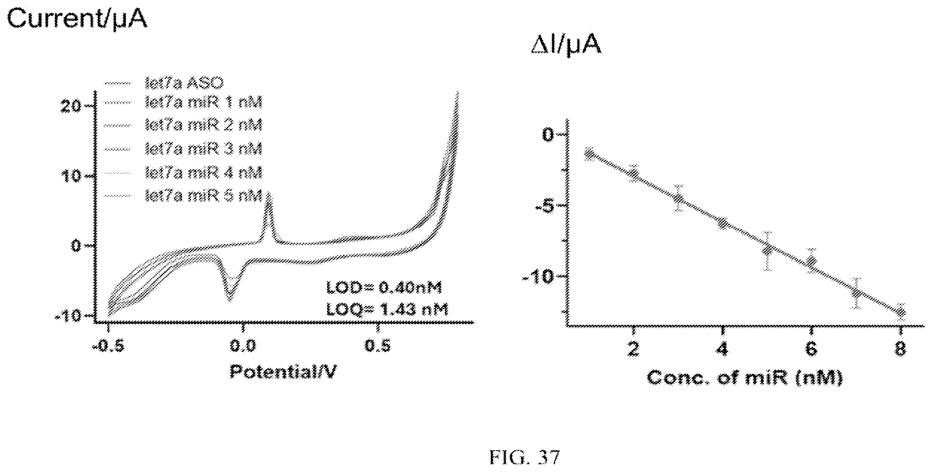

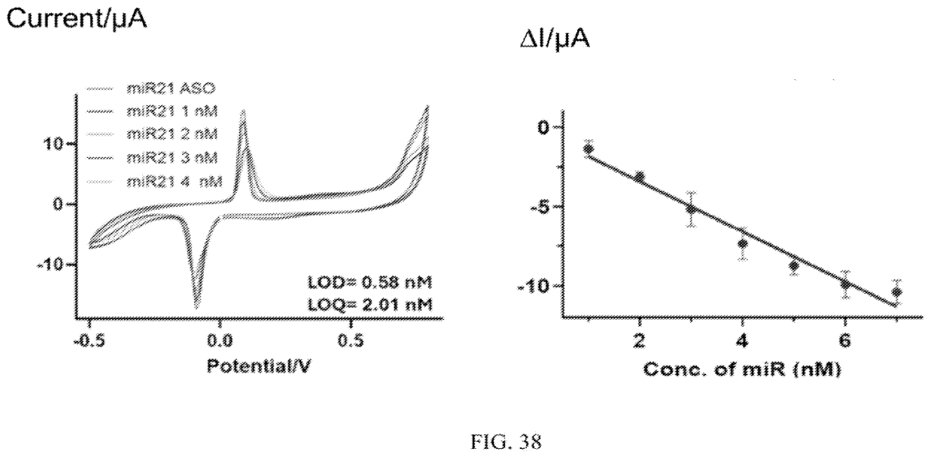

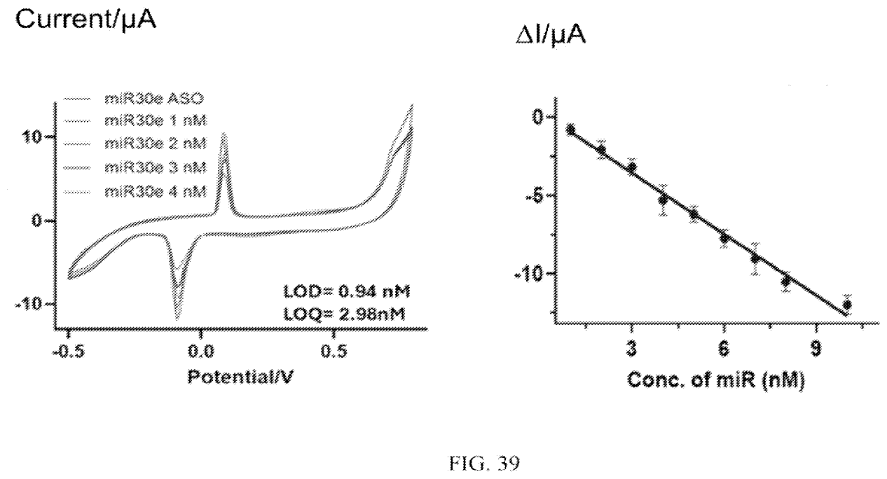

The hybridization of miR and anti-miR oligos was indirectly detected by monitoring changes in CV signals of guanine oxidation and electron transfer. MB attached to ant-miR ASOs moved away from the electrode upon miRNA binding, reducing electron transfer efficiency and current (FIG. 6). A gradual decrease in current (AI) was observed with increasing miRNA concentrations for miR-30e, miR-21, and let-7a (FIGS. 37-39). For miR-21, the current decreased linearly, with an LOD of 0.4-1 nM and LOQ of 1.4-3 nM. Similar trends were noted for all three miRNAs, with slight peak shifts. In DPV, a linear current increase at low concentrations (5-80 nM) was observed, attributed to potential pulses enhancing current. A new peak suggested a redox-active ASO-MB-miRNA complex. However, after multilayer electrode fabrication, DPV signals were less responsive at low concentrations than CV. Also, for miR21, the differential pulse voltammetry results were insignificant. DPV offered good sensitivity but was less effective with increased surface activity, making CV the preferred technique. Enhanced electrode functionalization improved electron transfer kinetics and active surface area, enabling stronger miRNA-ASO interactions and more pronounced redox signals in CV. Consequently, further assessments were conducted using CV only. The complex COF-polymer-anti-miR-based platform coupled with CV measurements demonstrated excellent selectivity and recovery in detecting these three miRNAs. Selectivity was validated through experiments using non-specific targets (random oligonucleotides, miR-320b and miR-16-5p), which showed no significant current output, confirming the strong hybridization affinity between the anti-miRNA probe and its complementary target. Recovery was assessed by spiking known concentrations of miRNAs into artificial saliva, yielding recoveries ranging from ˜96% to 103%, highlighting the sensor's accuracy and minimal interference from the matrix and similar analytes. These findings affirm the sensor's reliability for miRNA quantification in complex biological samples.

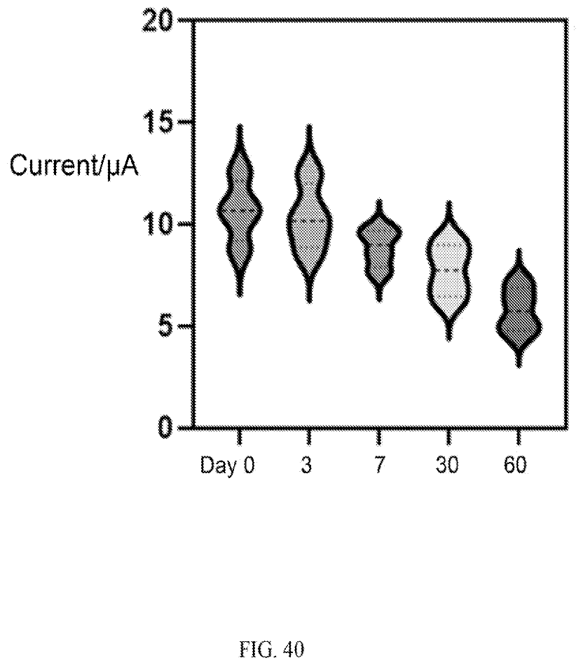

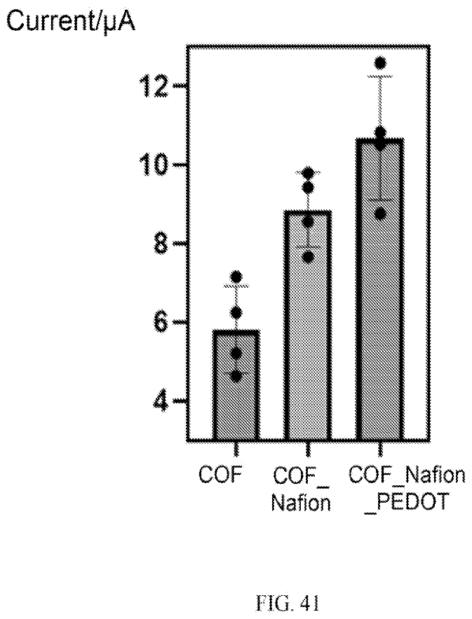

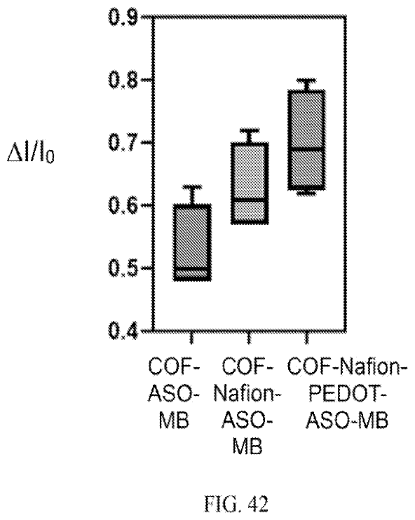

Results—Efficacy of Nafion/PEDOT with COF-Streptavidin system: Nafion and PEDOT:PSS were integrated with the COF layer in electrode strips to enhance cyclic voltammetry (CV) signals, crucial for detecting low salivary miRNA levels in mild traumatic brain injury (mTBI) patients. Nafion, a sulfonated fluoropolymer, was mixed with COF to improve ionic conductivity, mechanical strength, and stability, maintaining electrode functionality for 7 days at 4° C. and up to 60 days post-fabrication (FIG. 40). Nafion-COF electrodes outperformed COF-only surfaces in ASO or anti-miR conjugation via streptavidin-biotin chemistry, enhancing electrical output and facilitating proton/cation transport (FIGS. 41 and 42). The results confirm that Nafion, along with modified COF, acts as an ion exchange membrane, facilitating the transport of protons (H+) or other cations. This enhances the conductivity of the electrode and improves charge transfer processes.

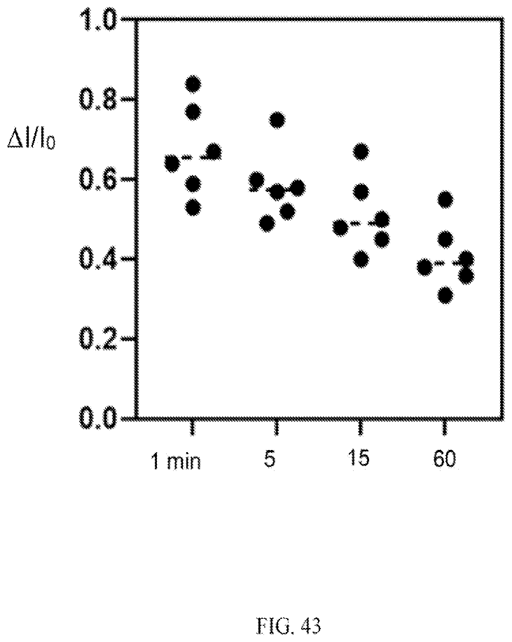

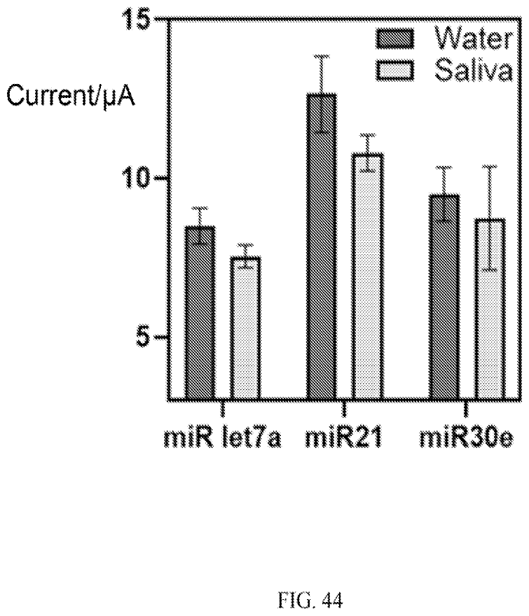

PEDOT:PSS, a conductive polymer, further increased sensor conductivity, stability, and flexibility, amplifying CV signals and reducing overpotential for MB redox reactions (FIGS. 41 and 42). Its porous structure expanded the electrode's effective surface area, enabling more MB-tagged ASOs to hybridize with miRNAs, improving signal strength and reproducibility (FIG. 43). The combined Nafion-PEDOT system ensured consistent electrochemical properties over multiple cycles, providing reliable detection of ultra-low miRNA concentrations (picomolar levels). This PEDOT/Nafion-COF-Streptavidin-ASO platform significantly enhanced electrochemical output, electron transfer, and sensitivity, making it a robust tool for salivary miRNA detection in mTBI patients. Tests in artificial saliva confirmed some matrix effects but maintained strong quantifiable signals (FIG. 44). This high-performance platform demonstrates excellent potential for electrochemical salivary miRNA sensors.

Results—mTBI salivary miRNA Detection from mTBI patient Samples: From previous UV-Vis and fluorescence spectroscopic measurements with artificial saliva, it was eminent that anti-miR ASOs exhibit minimal background interference from salivary components. After establishing the electrochemical detection method, saliva samples were collected from mTBI patients and healthy individuals (denoted as negative samples). The miRNA was extracted from the saliva through the standard miRNA extraction protocol, where the miRNA concentration in saliva was concentrated 10 folds, which was accounted for while performing the calculations. The results observed from electrochemical sensors were compared with PCR data to validate the presence of miRNAs in those samples. The details of PCR primers are provided in Table 4.

TABLE 4

| miR | Corresponding Primer sequence |
|---|---|
| hsa-miR-532-5p | 5′CAUGCCUUGAGUGUAGGACCGU |
| hsa-miR-151a-5p | 5′UCGAGGAGCUCACAGUCUAGU |
| hsa-miR-30a-5p | 5′UGUAAACAUCCUCGACUGGAAG |
| hsa-miR-181a-5p | 5′AACAUUCAACGCUGUCGGUGAGU |
| hsa-miR-708-5p | 5′AAGGAGCUUACAAUCUAGCUGGG |
| hsa-miR-148b-3p | 5′UCAGUGCAUCACAGAACUUUGU |
| hsa-let-7e-5p | 5′UGAGGUAGGAGGUUGUAUAGUU |
| hsa-miR-145-5p | 5′GUCCAGUUUUCCCAGGAAUCCCU |
| hsa-miR-320b | 5′AAAAGCUGGGUUGAGAGGGCAA |
| hsa-miR-16-5p | 5′UAGCAGCACGUAAAUAUUGGCG |

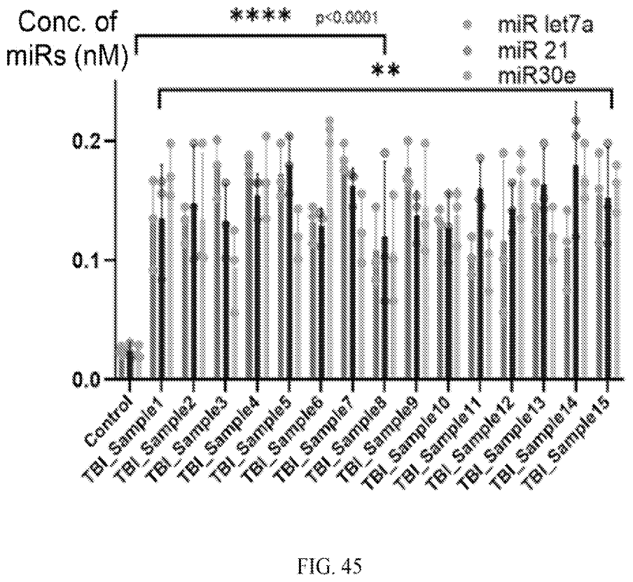

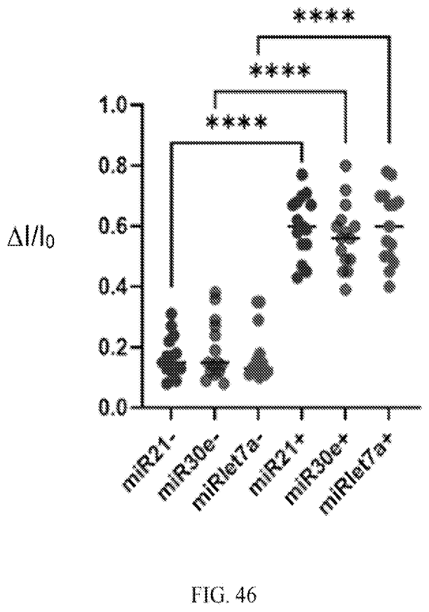

Results—Sensitivity and Specificity in Saliva Samples: Picomolar quantities of miRNA were successfully detected in saliva samples from patients with mild traumatic brain injury (mTBI). As miRNA extraction step concentrates the miRNA amount in samples, the picomolar concentrations in saliva were measured as nanomolar concentrations in the final extracted miRNA samples and the concentration factor was calculated. The concentrations of corresponding miRNAs were measured by cyclic voltammetry, and the values were calculated from previously obtained standard curves depicted for the corresponding miRNAs' Cyclic voltammetry data (FIG. 45). 2way ANOVA was performed for the grouped clinical samples. The electrochemical data correlated significantly between control and positive samples (****, p<0.0001), validating the sensor's accuracy (FIG. 46).

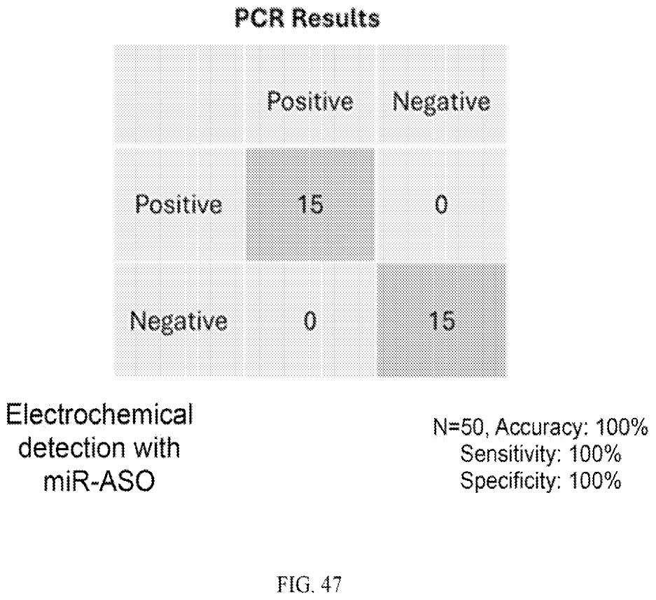

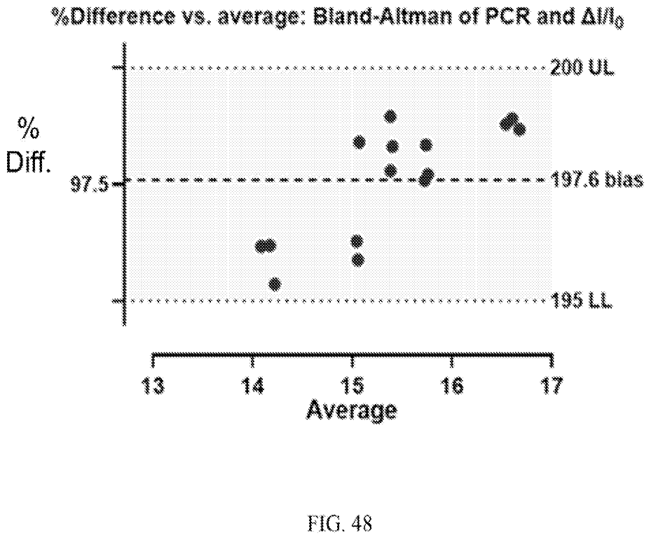

The miR let7a, mir21, and miR30e positive samples showed significant differences with negative clinical samples when measured in cyclic voltammetry and calculated as ΔI/I₀, where I₀ was the initial current output before the addition of sample and ΔI is the current (I) difference between before and addition of the sample (FIG. 46). The sensor demonstrated 100% sensitivity and specificity, accurately assessing 30 patient samples (15 positive and 15 negative). The confusion matrix in FIG. 47 shows the accuracy of the electrochemical detection and their correlation with PCR results. The Bland-Altman plot analysis (FIG. 48) was performed between ΔI/I₀ and PCR Cₜ values for the 15 positive mTBI clinical samples, and it was measured as % difference. The % difference was range bound within an upper limit of 200 and a lower limit of 195 for all the samples, which signifies the significance of the electrochemical detection compared to PCR results.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of the apparatus and process and/or utilization and methods of making and using the same have been discussed and illustrated herein, it is to be distinctly understood that the invention is not limited thereto but may be otherwise variously embodied and practiced within the scope of the following claims.

1-2. (canceled)

3. An apparatus for detecting a mild traumatic brain injury from a sample, the apparatus comprising:
an electrode comprising a polymeric layer coated on a surface of the electrode, wherein the polymeric layer includes at least one conductive polymer, and
one or more sensing probes immobilized on the surface of the electrode, wherein the one or more sensing probes comprise:
a first sensing probe having a sequence that is complementary to a first target gene sequence correlating to the mild traumatic brain injury, and
a second sensing probe having a sequence that is complementary to a second target gene sequence correlating to the mild traumatic brain injury,
wherein a first end of the one or more sensing probes is functionalized with a molecule configured to facilitate immobilization to the surface of the electrode, and wherein a second end of the one or more sensing probes is functionalized with an electrochemical reporter configured to produce a measurable signal,
wherein the first target gene sequence and the second target gene sequence are different sequences selected from the group consisting of UGAGGUAGUAGGUUGUAUAGU, UGUAAACAUCCUUGACUGGAAG, and UAGCUUAUCAGACUGAUGUUGA.

4. (canceled)

5. The apparatus of claim 3, wherein the one or more sensing probes further comprise a third sensing probe having a sequence that is complementary to a third target gene sequence correlating to the mild traumatic brain injury, and
wherein the first target gene sequence, the second target gene sequence, and the third target gene sequence are different sequences selected from the group consisting of UGAGGUAGUAGGUUGUAUAGU, UGUAAACAUCCUUGACUGGAAG, and UAGCUUAUCAGACUGAUGUUGA.

6. The apparatus of claim 3, wherein the molecule is biotin.

7. The apparatus of claim 3, wherein the electrochemical reporter is methylene blue.

8. The apparatus of claim 3, wherein the at least one conductive polymer is selected from the group consisting of polystyrene sulfonate, poly(3,4-ethylenedioxythiophene), polypyrrole, polyamine, and combinations thereof.

9. The apparatus of claim 3, wherein the electrode further includes a linking compound covalently attached to the polymeric layer and configured to facilitate immobilization of the one or more sensing probes to the surface of the electrode.

10. The apparatus of claim 9, wherein the linking compound is streptavidin.

11. The apparatus of claim 9, wherein the polymeric layer further includes at least one monomer polymerizable with the at least one conductive polymer.

12. The apparatus of claim 11, wherein the at least one monomer is functionalized with carboxylic acid groups configured to facilitate attachment of the linking compound to the polymeric layer.

13. The apparatus of claim 11, wherein the at least one monomer is a thiophene-based monomer functionalized with carboxylic acid groups configured to facilitate attachment of the linking compound to the polymeric layer.

14-15. (canceled)

16. A method for detecting a mild traumatic brain injury, the method comprising:
collecting a sample comprising nucleic acid from a subject;
providing an apparatus comprising:
an electrode comprising a polymeric layer coated on a surface of the electrode, wherein the polymeric layer includes at least one conductive polymer, and
one or more sensing probes immobilized on the surface of the electrode, wherein the one or more sensing probes comprise:
a first sensing probe having a sequence that is complementary to a first target gene sequence correlating to the mild traumatic brain injury, and
a second sensing probe having a sequence that is complementary to a second target gene sequence correlating to the mild traumatic brain injury,
wherein the one or more sensing probes have a sequence that is complementary to a target gene sequence correlating to the mild traumatic brain injury, wherein a first end of the one or more sensing probes is functionalized with a molecule configured to facilitate immobilization to the surface of the electrode, and wherein a second end of the one or more sensing probes is functionalized with an electrochemical reporter configured to produce a measureable signal,
wherein the first target gene sequence and the second target gene sequence are different sequences selected from the group consisting of UGAGGUAGUAGGUUGUAUAGU, UGUAAACAUCCUUGACUGGAAG, and UAGCUUAUCAGACUGAUGUUGA; and
applying the sample to the apparatus.

17. (canceled)

18. The method of claim 16, wherein the one or more sensing probes further comprise a third sensing probe having a sequence that is complementary to a third target gene sequence correlating to the mild traumatic brain injury, and
wherein the first target gene sequence, the second target gene sequence, and the third target gene sequence are different sequences selected from the group consisting of UGAGGUAGUAGGUUGUAUAGU, UGUAAACAUCCUUGACUGGAAG, and UAGCUUAUCAGACUGAUGUUGA.

19-20. (canceled)

21. An apparatus for detecting a mild traumatic brain injury from a sample, the apparatus comprising:
an electrode comprising a first polymeric layer, a covalent organic framework (COF) layer, and a second polymeric layer deposited on a surface of the electrode; and
one or more sensing probes immobilized on the surface of the electrode, wherein the one or more sensing probes comprise:
a first sensing probe having a sequence that is complementary to a first target gene sequence correlating to the mild traumatic brain injury, and
a second sensing probe having a sequence that is complementary to a second target gene sequence correlating to the mile traumatic brain injury,
wherein the one or mor sensing probes have a sequence that is complementary to a target gene sequence correlating to the mild traumatic brain injury,
wherein a first end of the one or more sensing probes is functionalized with a molecule configured to facilitate immobilization to the surface of the electrode, and wherein a second end of the one or more sensing probes is functionalized with an electrochemical reporter configured to produce a measureable signal,
wherein the first target gene sequence and the second target gene sequence are different sequences selected from the group consisting of UGAGGUAGUAGGUUGUAUAGU, UGUAAACAUCCUUGACUGGAAG, and UAGCUUAUCAGACUGAUGUUGA.

22. (canceled)

23. The apparatus of claim 21, wherein the one or more sensing probes further comprise a third sensing probe having a sequence that is complementary to a third target gene sequence correlating to the mild traumatic brain injury, and
wherein the first target gene sequence, the second target gene sequence, and the third target gene sequence are different sequences selected from the group consisting of UGAGGUAGUAGGUUGUAUAGU, UGUAAACAUCCUUGACUGGAAG, and UAGCUUAUCAGACUGAUGUUGA.

24. The apparatus of claim 21, wherein the molecule is biotin.

25. The apparatus of claim 21, wherein the electrochemical reporter is methylene blue.

26. The apparatus of claim 21, wherein the polymeric layer includes at least one conductive polymer.

27. The apparatus of claim 26, wherein the at least one conductive polymer is selected from the group consisting of polystyrene sulfonate, poly(3,4-ethylenedioxythiophene), polypyrrole, polyamine, and combinations thereof.

28. The apparatus of claim 21, wherein the COF layer is positioned between the first polymeric layer and the second polymeric layer.

29. The apparatus of claim 21, wherein the electrode further includes a linking compound covalently attached to the COF layer and configured to facilitate immobilization of the one or more sensing probes to the surface of the electrode.

30. The apparatus of claim 29, wherein the linking compound is streptavidin.

31. The apparatus of claim 29, wherein the COF layer is functionalized with epoxy groups configured to facilitate attachment of the linking compound to the COF layer.

32. The method of claim 16, wherein the apparatus comprises:
an electrode comprising a first polymeric layer, a covalent organic framework (COF) layer, and a second polymeric layer deposited on a surface of the electrode.

33-36. (canceled)


SEQUENCE LISTING (3 sequences)

SEQ ID NO: 1 - RNA, 22 nt, unidentified
  source 1..22: mol_type = genomic RNA
tgaggtagtaggttgtatagtt

SEQ ID NO: 2 - RNA, 22 nt, unidentified
  source 1..22: mol_type = genomic RNA
tgtaaacatccttgactggaag

SEQ ID NO: 3 - RNA, 22 nt, unidentified
  source 1..22: mol_type = genomic RNA
tagcttatcagactgatgttga